SEQUENCE LISTING (32 sequences)

SEQ ID NO: 1 - DNA, 39 bp, Artificial Sequence
  source: /note=“Description of Artificial Sequence Synthetic primer”
ctatatggccccagcatgaggcctctgtttgatttctcc

SEQ ID NO: 2 - DNA, 39 bp, Artificial Sequence
  source: /note=“Description of Artificial Sequence Synthetic primer”
ggagaaatcaaacagaggcctcatgctggggccatatag

SEQ ID NO: 3 - RNA, 44 nt, Artificial Sequence
  source: /note=“Description of Artificial Sequence Synthetic oligonucleotide”
cgacuggagcacgaggacacugacauggacugaaggaguagaaa

SEQ ID NO: 4 - RNA, 19 nt, Artificial Sequence
  source: /note=“Description of Artificial Sequence Synthetic oligonucleotide”
augccuuuguggaacuaua

SEQ ID NO: 5 - RNA, 19 nt, Artificial Sequence
  source: /note=“Description of Artificial Sequence Synthetic oligonucleotide”
uauaguuccacaaaggcau

SEQ ID NO: 6 - RNA, 19 nt, Artificial Sequence
  source: /note=“Description of Artificial Sequence Synthetic oligonucleotide”
gcaugcgaccucuguuuga

SEQ ID NO: 7 - RNA, 19 nt, Artificial Sequence
  source: /note=“Description of Artificial Sequence Synthetic oligonucleotide”
ucaaacagaggucgcaugc

SEQ ID NO: 8 - RNA, 19 nt, Artificial Sequence
  source: /note=“Description of Artificial Sequence Synthetic oligonucleotide”
ggaugacugaguaccugaa

SEQ ID NO: 9 - RNA, 19 nt, Artificial Sequence
  source: /note=“Description of Artificial Sequence Synthetic oligonucleotide”
uucagguacucagucaucc

SEQ ID NO: 10 - RNA, 19 nt, Artificial Sequence
  source: /note=“Description of Artificial Sequence Synthetic oligonucleotide”
aaaaaaaaaaaaaaaaaaa

SEQ ID NO: 11 - RNA, 19 nt, Artificial Sequence
  source: /note=“Description of Artificial Sequence Synthetic oligonucleotide”
gaagcgucuucuaauaauu

SEQ ID NO: 12 - RNA, 19 nt, Artificial Sequence
  source: /note=“Description of Artificial Sequence Synthetic oligonucleotide”
aauuauuagaagacgcuuc

SEQ ID NO: 13 - RNA, 19 nt, Artificial Sequence
  source: /note=“Description of Artificial Sequence Synthetic oligonucleotide”
uucuccgaacgugucacgu

SEQ ID NO: 14 - RNA, 19 nt, Artificial Sequence
  source: /note=“Description of Artificial Sequence Synthetic oligonucleotide”
acgugacacguucggagaa

SEQ ID NO: 15 - DNA, 19 bp, Artificial Sequence
  source: /note=“Description of Combined DNA/RNA molecule Synthetic oligonucleotide”
ggagaacagggtatgataa

SEQ ID NO: 16 - DNA, 19 bp, Artificial Sequence
  source: /note=“Description of Combined DNA/RNA molecule Synthetic oligonucleotide”
cctcttgtcccatactatt

SEQ ID NO: 17 - DNA, 19 bp, Artificial Sequence
  source: /note=“Description of Combined DNA/RNA molecule Synthetic oligonucleotide”
gcatgcggcctctgtttga

SEQ ID NO: 18 - DNA, 19 bp, Artificial Sequence
  source: /note=“Description of Combined DNA/RNA molecule Synthetic oligonucleotide”
cgtacgccggagacaaact

SEQ ID NO: 19 - DNA, 32 bp, Artificial Sequence
  source: /note=“Description of Artificial Sequence Synthetic oligonucleotide”
tcaaacagaggtcgcatgcctatagtgagtcg

SEQ ID NO: 20 - DNA, 32 bp, Artificial Sequence
  source: /note=“Description of Artificial Sequence Synthetic oligonucleotide”
gcatgcgacctctgtttgactatagtgagtcg

SEQ ID NO: 21 - DNA, 32 bp, Artificial Sequence
  source: /note=“Description of Artificial Sequence Synthetic oligonucleotide”
ggcgccccgccgcgccccgctatagtgagtcg

SEQ ID NO: 22 - DNA, 32 bp, Artificial Sequence
  source: /note=“Description of Artificial Sequence Synthetic oligonucleotide”
gcggggcgcggcggggcgcctatagtgagtcg

SEQ ID NO: 23 - DNA, 32 bp, Artificial Sequence
  source: /note=“Description of Artificial Sequence Synthetic oligonucleotide”
ttatcataccctgttctccctatagtgagtcg

SEQ ID NO: 24 - DNA, 32 bp, Artificial Sequence
  source: /note=“Description of Artificial Sequence Synthetic oligonucleotide”
ggagaacagggtatgataactatagtgagtcg

SEQ ID NO: 25 - DNA, 32 bp, Artificial Sequence
  source: /note=“Description of Artificial Sequence Synthetic oligonucleotide”
tcaaacagaggccgcatgcctatagtgagtcg

SEQ ID NO: 26 - DNA, 32 bp, Artificial Sequence
  source: /note=“Description of Artificial Sequence Synthetic oligonucleotide”
gcatgcggcctctgtttgactatagtgagtcg

SEQ ID NO: 27 - DNA, 25 bp, Artificial Sequence
  source: /note=“Description of Artificial Sequence Synthetic primer”
gttcatctgaagtttccagcctttg

SEQ ID NO: 28 - DNA, 23 bp, Artificial Sequence
  source: /note=“Description of Artificial Sequence Synthetic primer”
cgactggagcacgaggacactga

SEQ ID NO: 29 - DNA, 26 bp, Artificial Sequence
  source: /note=“Description of Artificial Sequence Synthetic primer”
ggacactgacatggactgaaggagta

SEQ ID NO: 30 - DNA, 32 bp, Artificial Sequence
  source: /note=“Description of Artificial Sequence Synthetic primer”
tccctttggcagtaaatagctgattcgaccat

SEQ ID NO: 31 - DNA, 30 bp, Artificial Sequence
  source: /note=“Description of Artificial Sequence Synthetic primer”
aagtcccttctccagtccatggaagaccag

SEQ ID NO: 32 - DNA, 33 bp, Artificial Sequence
  source: /note=“Description of Artificial Sequence Synthetic primer”
ctttggcagtaaatagctgattcgaccatttgc

(19) United States
(12) Patent Application Publication
(10) Pub. No.: US 2011/0184045 A1
(43) Pub. Date: Jul. 28, 2011

(54) SILENCNG AND RIG-I ACTIVATION BY DUAL FUNCTION OLIGONUCLEOTIDES

(76) Inventor: Gunther Hartmann, Cambridge, MA (US)

(21) Appl. No.: 13/002,101

(22) PCT Filed: Jun. 30, 2009
(86) PCT No.: PCT/US09/49194

§ 371 Date: Mar. 28, 2011

Related U.S. Application Data
(60) Provisional application No. 61/076,986, filed on Jun. 30, 2008.

(51) Int. Cl. A61K 31/713; C12Q 1/68; A61P 35/00

(52) U.S. Cl. 514/44 A; 435/6.13

(57) ABSTRACT

The invention describes a method of determining whether a double stranded RNA (dsRNA) silences gene expression in a cell in vivo by an RNA interference (RNAi) mechanism by performing 5′-rapid amplification of cDNA ends (5′RACE) to detect the cleavage site of the mRNA in the RNA sample.

### CROSS REFERENCE TO RELATED APPLICATIONS

This application claims the benefit of U.S. Provisional Application No. 61/076,986, filed Jun. 30, 2008, the entire disclosure of which is hereby incorporated by reference in its entirety for all purposes.

### STATEMENT REGARDING FEDERALLY SPONSORED RESEARCH OR DEVELOPMENT

Not applicable.

### BACKGROUND OF THE INVENTION

1. Field of the Invention

The invention relates to methods and compositions for silencing and RIG-1 activation, and a method of determining whether a double stranded RNA (dsRNA) silences gene expression in a cell in vivo by an RNA interference (RNAi) mechanism by performing 5′-rapid amplification of cDNA ends (5′RACE) to detect the cleavage site of the mRNA in the RNA sample.

2. Description of the Related Art

Cellular transformation and progressive tumor growth result from an accumulation of mutational and epigenetic changes that alter normal cell proliferation and survival pathways 1. Tumor pathogenesis is accompanied by a process called cancer immunoediting, a temporal transition from immune-mediated tumor elimination in early phases of tumor development to immune escape of established tumors. The interferons (IFNs) have emerged as central coordinators of these tumor-immune-system interactions 2. Due to genetic and epigenetic plasticity, tumors tend to evade single-targeted therapeutic approaches such as specific kinase inhibitors used to control survival of tumor cells 3; tumors even evade immunotherapies that by definition are capable of targeting multiple tumor antigens 4. There are good reasons to believe that a combinatorial approach that suppresses tumor cell survival and at the same increases immunogenicity of tumor cells may lead to more effective tumor treatments 5, 6.

Short double-stranded (ds) RNA oligonucleotides offer excellent properties for such a combinatorial approach 7. The sequence of short dsRNA oligonucleotides can be selected to specifically silence individual key proteins responsible for tumor cell survival of different tumor entities 8; such RNA oligonucleotides (siRNA) make use of the mechanism of RNA interference (RNAi) that is present in any cell type including tumor cells 9. A distinct and independent biological property of RNA oligonucleotides can be the activation of immunoreceptors specialized for the detection of viral nucleic acids.

The RNA helicase RIG-I is one of two immunoreceptors that signal the presence of viral RNA in the cytosol of cells 10. Specifically, RIG-I detects RNA with a triphosphate group at the 5′ end. Formation of such 5′-triphosphate RNA by RNA polymerases in the cytosol of cells is characteristic for most negative strand RNA viruses 11, 12. Like the RNA interference machinery and the RNA-induced silencing complex (RISC), RIG-I is expressed in all cells. Sensing of 5′-triphosphate RNA via RIG-I signals two key antiviral responses: i) production of type I IFN and Th1 chemokines, and ii) apoptosis 13. Induction of type I IFN and apoptosis by 5′-triphosphate RNA (3pRNA) are not only the natural response to viral infection; both are highly desired biological activities for tumor therapy.

Since recognition of 3pRNA by RIG-I is largely independent of the 3′ RNA sequence, and, on the other hand, gene silencing is not affected by the presence of a triphosphate group at the 5′ end, both biological activities can be combined in one short dsRNA molecule. Such a short dsRNA molecule with triphosphate groups at the 5′ end (3p-siRNA) can be adapted to different tumor entities by targeting the gene silencing activity to corresponding key tumor survival factors. In the case of melanoma, a key molecule required for tumor cell survival is bcl-2. Bcl-2 was originally found in B cell lymphomas and is involved in regulation of the mitochondrial apoptosis pathway. Overexpression of bcl-2 is considered to be responsible for the extraordinary resistance of melanoma cells to chemotherapy 14-16.

### SUMMARY OF THE INVENTION

Two hallmarks of tumor development are increased tumor cell survival and immune escape. Genetic and epigenetic plasticity allow tumors to evade single-targeted treatments. Here we direct short interfering RNA (siRNA) containing triphosphate groups at the 5′ ends (3p-siRNA) against melanoma. The 3p-siRNA used comprises two distinct and independent functional activities in one molecule: silencing of anti-apoptotic bcl-2, and activation of the cytosolic helicase RIG-I. Systemic treatment with bcl-2-specific 3p-siRNA elicited strong anti-tumor activity in a metastatic melanoma model. Like TLR agonists, RIG-I ligation by 3p-siRNA activated innate immune cells such as dendritic cells; unlike TLR agonists, activation of RIG-I directly induced a type I IFN response and apoptosis in murine and human tumor cells; RIG-1-induced apoptosis of tumor cells synergized with apoptosis induced by siRNA-mediated silencing of bcl-2 in tumor cells. In vivo, these mechanisms acted in concert to provoke massive apoptosis of tumor cells in lung metastases. The overall therapeutic activity of 3p-siRNA in vivo required NK cells and type I IFN and was associated with downregulation of bcl-2 in metastatic tumor cells in vivo on a single cell level. Together, 3p-siRNA represents a novel single molecule-based combinatorial approach in which RIG-I activation on both the immune- and the tumor cell level corrects immune ignorance and in which gene silencing is used to correct key molecular events that govern tumor cell survival.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates that 3p-2.2 siRNA potently silences bcl-2 expression and reduces metastatic growth of B16 melanoma cells in the lungs. (a) left panel: B16 cells were seeded in 24-well plates at a confluency of 50%, B16 cells were transfected with the selected chemically synthesized siRNAs (anti-bcl-2 2.1, anti-bcl-2 2.2 and anti-bcl-2 2.3) at 1 μg/ml. 48 hours after transfection, protein expression of murine bcl-2 was analyzed by Western blot. A non-silencing siRNA (Control-RNA=Ctrl.) served as negative control. Right panel: siRNA 2.2 (OH-2.2) was in vitro transcribed. This generates a siRNA with the same sequence as the synthetic siRNA which bears an additional 5′-triphosphate group (termed 3p-2.2). An in vitro transcribed 3p-RNA with an unspecific GC-rich sequence (termed 3p-GC) served as negative control. 48 hours after transfection of the chemically synthesized siRNAs (Ctrl.; OH-2.2) and of the in vitro transcribed 3p-siRNA (3p-GC and 3p-2.2) protein expression of murine bcl-2 was analyzed by Western blot. One representative experiment of four is shown. (b) Left panel: 5′-RACE demonstrating RNA interference induced by 3p-siRNA. Black arrows mark the 5′ RACE-PCR amplification product showing the predicted product of RNA-interference (RNAi) in the siRNA-treated B16 cells. Right panel: schematic diagram showing the position of the predicted siBcl-2 cleavage site relative to nested primers used for PCR amplification of the cleavage fragment. (c) Therapeutic efficacy of two distinct 5′-triphosphate siRNAs in a murine lung metastases model. Groups of five C57BL/6 mice were challenged with 4×10⁵ B16 cells and treated intravenously on days 3, 6, and 9 with 50 μg of OH-2.2 (bcl-2 silencing activity), Control-RNA (no silencing activity), 3p-2.2 (bcl-2 silencing and RIG-I activation) or 3p-GC(RIG-I activation, but no silencing activity), All RNAs were coupled to jetPEI. Tumor growth was assessed after 14 days by measuring the weight of the lungs. The mean lung weights (sum of both lungs) of five individual mice are indicated by the columns. The lung weight of healthy mice ranges between 0.20 and 0.24 g (P**<0.01 between 3p-2.2 and Ctrl., OH-2.2 and 3p-GC; n=5; Mann-Whitney U test).

FIG. 2 illustrates activation of type I IFNs and NK cells mediate the anti-tumor activity of bcl-2-specific immunostimulatory 3p-siRNA in vivo. (a) Groups of wild-type (WT) mice, IFN-α-receptor 1-deficient (IFNAR^{-/-}) or toll-like receptor 7-deficient (TLR7^{-/-}) mice were treated with 3p-2.2 as described in FIG. 1c. Tumor growth was assessed on day 14 by counting the number of macroscopically visible melanoma metastases on the lung surfaces. Shown is the number of metastases in individual C57BL/6 mice. The mean number of metastases is indicated by the horizontal line. Left panel: P*<0.05 between 3p-2.2 and Control-RNA-treated in WT mice; n=4; Mann-Whitney U test; middle panel: Effect of 3p-2.2 in IFNAR^{-/-} mice: P*>0.05 between 3p-2.2 and Control-RNA-treated mice (n=4); right panel: Effect of 3p-2.2 in TLR7^{-/-} mice: P*<0.05 between 3p-2.2 and Control-RNA-treated mice (n=4) (b) Effect of antibody-based depletion of CD8 T cells and NK cells on the therapeutic anti-tumor efficacy of 3p-2.2 in C57BL/6 wildtype mice (P*<0.05; n=5)).

FIG. 3 illustrates Bcl-2-specific immunostimulatory 3p-siRNA induces innate immune responses and apoptosis in vitro. (a) GMCSF-derived conventional DC (cDC) were transfected with 1 μg/ml of OH-2.2, 3p-GC or 3p-2.2. After 24 h IFN-α production was quantified in the supernatant by ELISA. Data are shown as means±SEM of two independent experiments. (b) B16 cells and murine fibroblasts (NIH-3T3 cells) were seeded in 24-well plates and transfected with an IFN-β promoter reporter construct containing luciferase. 24 h ells were transfected with OH-2.2, 3p-GC or 3p-2.2 (1 μg/ml each). After 16 h cells were analyzed for IFN-β luciferase activity. Data are shown as mean±SEM of two independent experiments. (c) B16 cells were stimulated with 3p-2.2 (1 μg/ml) or murine IFN-13 (1,000 U/ml). After 8 h cells were analyzed by Western blot for RIG-I expression. HEK293 cells overexpressing RIG-I served as positive control. One representative experiment of two is shown. (d) B16 cells were transfected with indicated RNAs (1 μg/ml each). 24 h after transfection cells were analyzed by flow cytometry for apoptosis. Apoptotic cells were defined as Annexin-V positive and propidium iodide negative cells. Results are shown as mean±SEM of four independent experiments (P**<0.01 3p-2.2 versus OH-2.2 and Control-RNA (Ctrl).; P*<0.05 3p-GC versus OH-2.2 and Control-RNA.; t-test) (e) B16 cells were transfected with OH-2.2, 3p-GC or 3p-2.2 in combination with siRNA specific for RIG-I or Control-RNA. Cells were analyzed for apoptosis 24 later. Data are shown as mean±SEM of three independent experiments (P**<0.01 between Control-RNA 3p-2.2 versus RIG-I siRNA 3p-2.2; t-test). (f) Murine fibroblasts (NIH3T3) were treated and analyzed for apoptosis as described in (d). Staurosporine was used as a positive control. Results are shown as means±SEM of two independent experiments.

FIG. 4 illustrates Bcl-2-specific gene silencing and activation of the innate immune system synergistically promotes tumor cell apoptosis in vivo. (a) C57BL/6 mice were injected with Control-RNA, OH-2.2, 3p-GC or 3p-2.2 (50 μg/Mouse) as described. Sera were collected after 6 h and IFN-α levels determined by ELISA. Data are shown as mean±SEM of six independent experiments. (b) Infiltration of NK cells in single cell suspensions of metastatic lungs was analyzed by flow cytometry. Results are presented as mean numbers of NK-1.1 positive cells±SEM (P*<0.05 between 3p-2.2 and Control-RNA-treated mice; P*<0.05 between 3p-GC and Control RNA-treated mice; n=4). (c) Activation of NK cells in single cell suspensions of metastatic lungs was analyzed by flow cytometry for CD69. Results are presented as mean percentage of cells±SEM (P*<0.05 between OH-2.2 and Control-RNA-treated mice; P**<0.01 between 3p-2.2, 3p-GC and Control-RNA treated mice; n=4; t-test). (d) Bcl-2 expression of B16 tumor cells in single cell suspensions of metastatic lungs was quantified by gating on HMB45 positive cells. Depicted is the mean fluorescence intensity (MFI)±SEM (P*<0.05 between 3p-2.2 and Control-RNA and 3p-GC treated mice; P*<0.05 between OH-2.2 and Control RNA-treated mice; n=4; t-test). (e) In vivo 5′-RACE analysis of RNA extracted from metastatic lungs demonstrating that silencing of bcl-2 mRNA is due to RNAi-mediated mRNA cleavage 24 h after treatment with the indicated siRNAs. Black arrows mark the 5′ RACE-PCR amplification product showing the predicted product of RNAi in the siRNA-treated animals. (f) Groups of five C57BL/6 mice were treated as described. Samples of lungs were fixed in ethanol, embedded in paraffin and analyzed for apoptotic tumor cells. Upper panel: Melanoma cells were visualized in lung tissue sections by HMB45 immunohistochemistry (black arrows). Middle and lower panel: Apoptotic cells were detected within metastases by the transferase-mediated dUTP nick end-labeling (TUNEL) (black arrows). Representative sections of one experiment with five mice/group are shown.

FIG. 5 illustrates Bcl-2-specific gene silencing contributes to 3p-siRNA induced inhibition of tumor growth and apoptosis. (a) B16 cells transduced with a codon-optimized Bcl-2 cDNA designed to rescue siRNA activity of siRNA 2.2 (Mut-B16) and control-transduced cells (WT-B16) were seeded in 12-well flat-bottom plates. At a confluency of 50-70% cells were transfected with the indicated siRNAs (1 μg/ml each). 24 h after transfection, protein expression of murine bcl-2 was analyzed by Western blot. (b) Left panel: WT-B16 or Mut-B16 cells were transfected with the indicated RNAs (1 μg/ml each). 48 h after transfection cells were analyzed by flow cytometry for the induction of apoptosis. Apoptotic cells were defined as Annexin-V positive and propidium iodide negative cells. Results are shown as mean percent of apoptotic cells±SEM of three independent experiments. Right panel: one representative dot plot of three independent experiments is shown. (c) Therapeutic anti-tumor efficacy of siRNAs OH-2.4 and 3p-2.4 against B16 melanoma metastases in the lungs. Groups of four C57BL/6 mice were challenged i.v. with 4×10⁵ B16 cells and treated with 50 μg each of the indicated siRNAs coupled to jetPEI. After 14 days the number of macroscopically visible melanoma metastases on the lung surfaces was counted (lower panel). (d) Groups of three C57BL/6 mice were challenged with 4×10⁵ WT-B16 or Mut-B16 and treated as described. After 14 days the number of macroscopically visible melanoma metastases was counted on the lung surfaces. * P<0.01.

FIG. 6 illustrates the efficacy of Bcl-2-specific 3p-siRNA can be extended to other models of tumorigenesis and to the human system in vitro. a) Groups of five CDK4^{R24C} mutant C57BL/6 mice were intracutaneously injected with approximately 105 viable melanoma cells derived from primary cutaneous melanomas of HGF×CDK4^{R24C} by serial transplantation. Mice were treated with intra- and peritumoral injections of 50 μg 3p-2.2 coupled to jetPEI on days 10, 16, 24 and 30. Control mice received PBS. Tumor growth was monitored twice weekly and tumor size calculated according to the formula Volume=(L×W²)×0.5 and expressed in mm³. Shown is the mean tumor volume of each group. ** P<0.01. (b) Groups of five Balb/c mice were injected with 2.5×105 C26 cells subcutaneously in the right flank. Mice were treated intravenously on days 6, 9, 12 and 15 with 50 μg each of the indicated siRNAs coupled to jetPEI. Tumor growth was monitored three times weekly and expressed as the product of the perpendicular diameters of individual tumors. Shown is the mean tumor area of each group. ** P<0.01. (c) Immunostimulatory efficacy and silencing of 3p-h2.2 in the human melanoma cell line 1205Lu. Cells were treated with the indicated siRNAs (1 μg/ml) and analyzed after 17 h. Immunostimulatory activity was accessed by measuring IFN-β RNA expression by quantitative RT-PCR (left panel). IFN-β RNA expression values were normalized to Hypoxanthine-phosphoribosyl-transferase (HPRT). The mean±SD of three independent experiments is shown. Bcl-2-silencing activity was accessed by immunoblotting (right panel). β-actin served as loading control. Blots are representative of three independent experiments. (d) The human metastatic melanoma cell line WM239A was transfected with siRNAs using Lipofectamine RNAiMAX (Invitrogen, Karlsruhe, Germany) at 1 μg/ml according to the manufacturers protocol. Apoptosis was determined 24 h after transfection by staining with FITC-conjugated Annexin-V and propidium iodide. A representative dot blot of three experiments is shown. (e) FACS analysis of apoptotic cell death in human melanoma cell lines derived from different tumor stages, i.e. WM793 and 1205Lu. Cells were treated with siRNAs as described in (c) and analyzed after 24 h. The mean±SD of three independent experiments is shown. (f) Cell viability of human melanoma cell lines, melanocytes and primary fibroblasts 24 h after transfection of 3p-h2.2. Viability was quantified as described 32. Four melanoma cell lines (1205Lu, WM278, WM793, WM239A) as well as melanocytes and fibroblasts from three different donors were measured. Viability of samples treated with control siRNA was set to 100%. The mean±SD of three independent experiments is shown for melanoma cell lines.

FIG. 7 is a schematic diagram of the potential anti-tumor mechanisms elicited by 3p-siRNA. 3p-2.2 contains two clearly distinct functional properties, a) gene silencing and b) RIG-I activation. 3p-2.2 is able to trigger the following distinct anti-tumor mechanisms: i) RIG-I is expressed in immune cells and non-immune cells including tumor cells; activation of RIG-I leads to direct (1) and indirect activation (2) of immune cell subsets (NK cells, CD8 and CD4 T cells), but also provokes innate responses directly in tumor cells (type I IFNs and chemokines) (3). ii) In addition, RIG-I activation directly induces apoptosis in melanoma cells (which are sensitive to RIG-I-mediated apoptosis) (4) and iii) silencing of bcl-2 induces apoptosis in cells that depend on bcl-2 overexpression (5). The activation of RIG-I in tumor cells may sensitize these cells for specific destruction by innate effector cells (6).

FIG. 8 illustrates that IFN-α production by 5′-triphosphate siRNA requires RIG-I in cDC, but not MDA-5. (a) Sorted pDC from Flt3-L-induced bone marrow cultures and GMCSF-derived cDCs were transfected with 1 μg/ml of 3p-2.2. In addition, B cells, NK cells and CD8 T cells were purified from spleens of wild-type mice (WT) and transfected with 1 μg/ml of 3p-2.2. After 24 h IFN-α production was quantified in the supernatant by ELISA. Data are shown as means±SEM of two independent experiments. (b) GMCSF-derived cDC of wild-type (WT), RIG-I- and MDA-5-deficient mice were transfected with 1 μg/ml of OH-2.2, 3p-GC, 3p-2.2, and Poly(I:C). After 24 h IFN-α production was quantified in the supernatant by ELISA. Data are shown as means±SEM of two independent experiments. (c) GMCSF-derived cDC of WT and TLR7-deficient mice were transfected with 1 μg/ml of OH-2.2, 3p-2.2 and CpG 2216 (3 μg/ml). After 24 h IFN-α production was quantified in the supernatant by ELISA. Data are expressed as the mean±SEM of two independent experiments.

FIG. 9 illustrates that. 5′-triphosphate siRNA leads to RIG-I dependent activation of murine B16 cells and Cardif-independent apoptosis. (a) B16 cells were treated with the indicated stimuli as described. IP-10 production was quantified in the supernatant by ELISA. Data are shown as means±SEM of two independent experiments. (b) B16 cells were treated with the indicated stimuli as described. After 24 h the number of MHC-I positive cells were determined by FACS-analysis. One representative histogram out of two independent experiments is shown. (c, d) B16 cells were transfected with synthetic siRNAs as described in material and methods. 24 h after transfection cells were stimulated with 3p-2.2 (1 μg/ml). 16 h after stimulation cells were analyzed for IFN-β luciferase reporter activity. Data are shown as means±SEM of three independent experiments.

FIG. 10 illustrates that 3 5′-triphosphate siRNA leads to cytokine secretion in vivo. C57BL/6 and TLR7−/− mice were treated with 3p-2.2 and OH-2.2. After 6 h mice were sacrificed and serum was analyzed for IFN-α (a), IL-12p40 (b) and IFN-γ (c) by ELISA. Data are shown as means±SEM of two independent experiments.

FIG. 11 illustrates that 4 5′-triphosphate siRNA enhances the production of serum cytokines in vivo. C57BL/6 mice were injected intravenously with increasing doses of 3p-2.2 (25, 50 or 75 μg/mouse). Serum was collected after 6 h. Cytokine levels of IFN-α (a) and IL-12p40 and IFN-γ (b) were determined by ELISA. (c) C57BL/6 mice were injected with 3p-2.2 and OH-2.2 and serum was collected 12 h, 24 h, and 48 h after injection. Serum cytokine levels of IFN-γ were determined by ELISA. Data are shown as means±SEM of two independent experiments. (d, e) C57BL/6 mice were treated with 3p-2.2 and OH-2.2 and blood was collected after 48 h and processed as EDTA plasma for measurement of (d) leucocytes (WBC) and platelets (PLT) (e). Data are shown as means±SEM of two independent experiments.

FIG. 12 illustrates that 5.5′-triphosphate siRNA activates immune cell subsets in vivo. C57BL/6 mice were injected with increasing doses of 3p-2.2 (25, 50 or 75 μg/mouse). Left panel: Spleen cells were isolated 48 h after injection and CD86 or CD69 expression was analyzed on pDC, mDC, NK cells, CD4 T cells and CD8 T cells by flow cytometry. Data are shown as means±SEM of two independent experiments. Right panel: Histograms of one representative experiment after stimulation with 50 μg 3p-2.2 is shown (grey bar, PBS treated control mice; white bar, 3p-2.2 treated mice).

FIG. 13 illustrates that 5′-triphosphate siRNA induces NK cell cytotoxicity independent of TLR7 (a) Activation of splenic NK cells isolated from 3p-2.2-injected wild-type, strictly depends on IFNAR, but not TLR7. WT, TLR7- or IFNAR-deficient mice were administered with 3p-2.2 (or control saline only) i.v. After 16 h, splenic NK cells were isolated with DX5 (anti-CD49b) microbeads and assayed for activation by flow cytometry. (b) WT and TLR7−/− were administered with OH-2.2, 3p-2.2 or PBS i.v. After 16 h, NK cells were isolated with DX5 (anti-CD49b) microbeads and NK cytotoxicity against B16 cells was measured by 51Cr release assay. YAC-1 cytotoxicity of splenic NK cells was tested at the same time since YAC-1 is known to be targets for NK cells.

FIG. 14 illustrates in vivo uptake and silencing activity of 5′-triphosphate siRNA in lung metastases. B16 cells were intravenously injected into C57BL/6 mice and 14 days after tumor inoculation, a single dose of FITC-labeled siRNA (100 μg) was administered retro-orbitally. After 6 h the mice were sacrificed and various tissues including lungs were excised and the uptake of FITC-labeled siRNA was analyzed by confocal microscopy. As expected, in the case of noncomplexed siRNAs no uptake was observed in lungs of healthy mice and in mice with lung metastases indicating the rapid and complete degradation of the FITC-labeled siRNA (upper panel, −PE1). In contrast, upon PE1 complexation intact siRNA was detected in high amounts in several tissues including liver and spleen (data not shown). Considerable amounts of FITC-labeled siRNA were detected in lungs of healthy mice, but also (although to a lower extent) in lung metastases of diseased mice (lower panel, +PE1). One representative out of two independent experiments after injection with 100 μg FITC-labeled siRNA is shown.

### DETAILED DESCRIPTION OF THE INVENTION

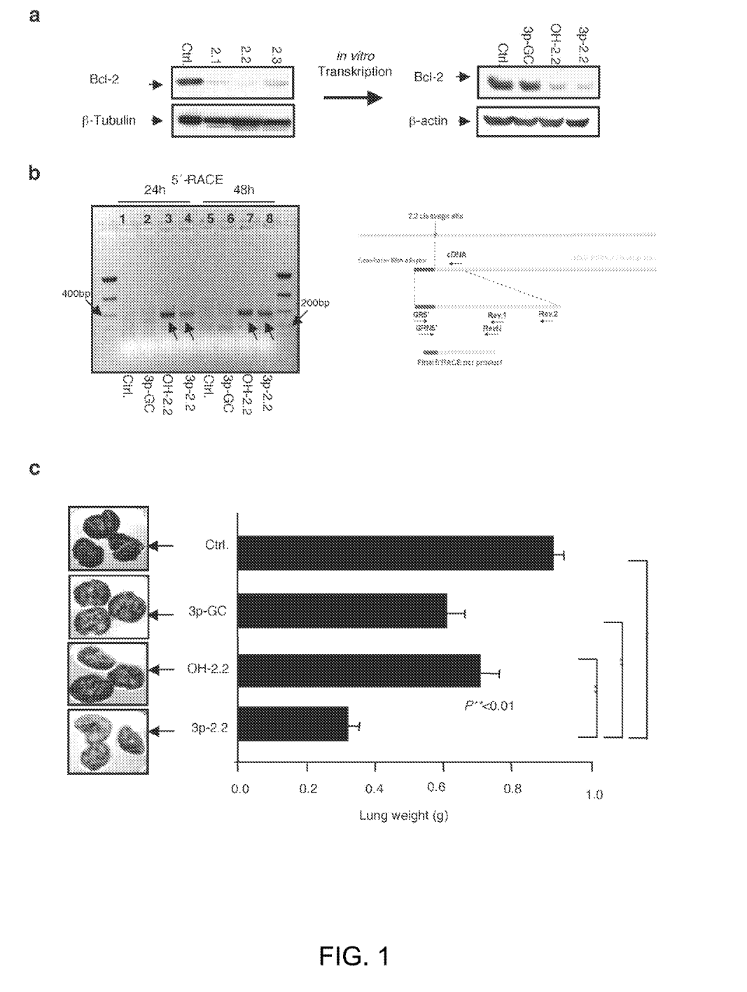

In order to test the feasibility of the 3p-siRNA approach for tumor therapy, we designed three synthetic siRNAs (anti-bcl-2.1, anti-bcl-2.2, anti-bcl-2.3) targeting different parts of murine bcl-2 mRNA (for a detailed list of all chemically synthesized RNA oligonucleotides see Table 1). The ability of these anti-bcl-2 siRNA sequences to downregulate bcl-2 protein was analyzed in murine B16 melanoma cells (FIG. 1a, left panel). Being the most effective, anti-bcl-2.2 siRNA was selected for subsequent experiments. Next, T7 RNA polymerase was used to generate siRNA that in addition to the anti-bcl-2 sequence (i.e. anti-bcl-2 siRNA) contains triphosphate groups attached to both 5′ ends (anti-bcl-2 3p-siRNA; for a detailed list of all in vitro transcription templates see Table 2). Anti-bcl-2 siRNA with 5′-triphosphate groups is termed 3p-2.2; the same siRNA sequence without 5′-triphosphate groups is termed OH-2.2. 3p-2.2 was equally effective as OH-2.2 in silencing bcl-2 gene expression (FIG. 1a, right panel). The control 3p-RNA with an unrelated RNA sequence (3p-GC) did not downregulate bcl-2 expression (FIG. 1a, right panel). We also confirmed that bcl-2 silencing is mediated by RNAi, as demonstrated by 5′ rapid amplification of cDNA ends (RACE) analysis and identification of the predicted cleavage site, exactly ten nucleotides from the 5′ end of the antisense strand of OH-2.2 and 3p-2.2. (FIG. 1b). RACE products were confirmed by sequencing (data not shown).

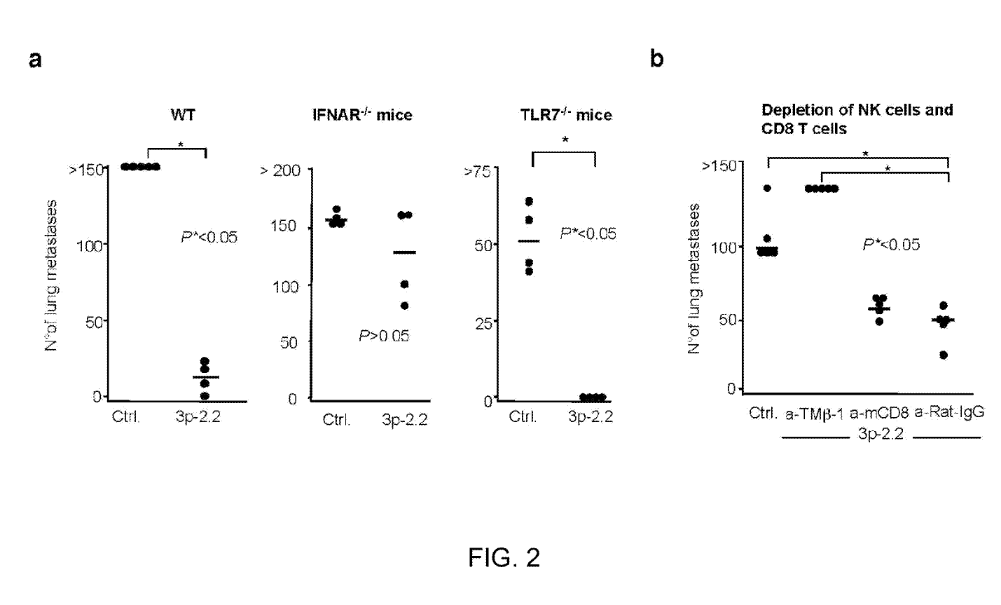

Next we examined the anti-tumor activity of bcl-2-specific 3p-siRNA (termed 3p-2.2) in the B16 melanoma lung metastases model in vivo. Following intravenous (i.v.) injection of B16 tumor cells on day 0, mice received i.v. injections of different RNA molecules on day 3, day 6 and day 9. On day 14, mice were sacrificed and growth of experimentally induced melanoma metastases assessed. 3p-GC, and a synthetic control RNA (Ctrl.) were used as negative controls. As shown in FIG. 1c, OH-2.2 (gene silencing activity but no RIG-I ligand activity expected) and 3p-GC(RIG-I ligand activity but no gene silencing activity expected) both inhibited the growth of melanoma metastases to a certain degree. Importantly however, 3p-2.2, which combines bcl-2-specific gene-silencing and immunostimulatory properties, displayed significantly enhanced therapeutic anti-tumor activity (P**<0.01 of 3p-2.2 compared to OH-2.2, 3p-GC or Ctrl.). 5′-triphosphate siRNA were specifically designed to stimulate the type I interferon system. Experiments in type I IFN receptor knockout mice (IFNAR−/−) confirmed that the observed anti-tumor activity of 3p-2.2 in vivo strongly depended on the presence of type I IFNs (FIG. 2a, middle panel). It has been reported that siRNA can be detected by TLR7 in a sequence dependent manner leading to the formation of type I IFN^{17,18}. We found that TLR7 was dispensable for the anti-tumor activity of 3p-2.2 in the B16 melanoma model (FIG. 2a, right panel). This indicated that TLR7-induced type I IFN production (in plasmacytoid dendritic cells) is not required and suggested that RIG-1-mediated 3p-2.2 recognition and type I IFN induction plays a dominant role. Depletion studies demonstrated that the anti-tumor activity of 3p-2.2 in the B16 melanoma model depended on NK cells but not CD8 T cells (FIG. 2b). Together these results confirm that both gene silencing (since the 3p control 3p-GC is significantly less active) and RIG-I (but not TLR7) dependent innate immunity contribute to the anti-tumor activity of 3p-2.2 in the B16 melanoma model in vivo.

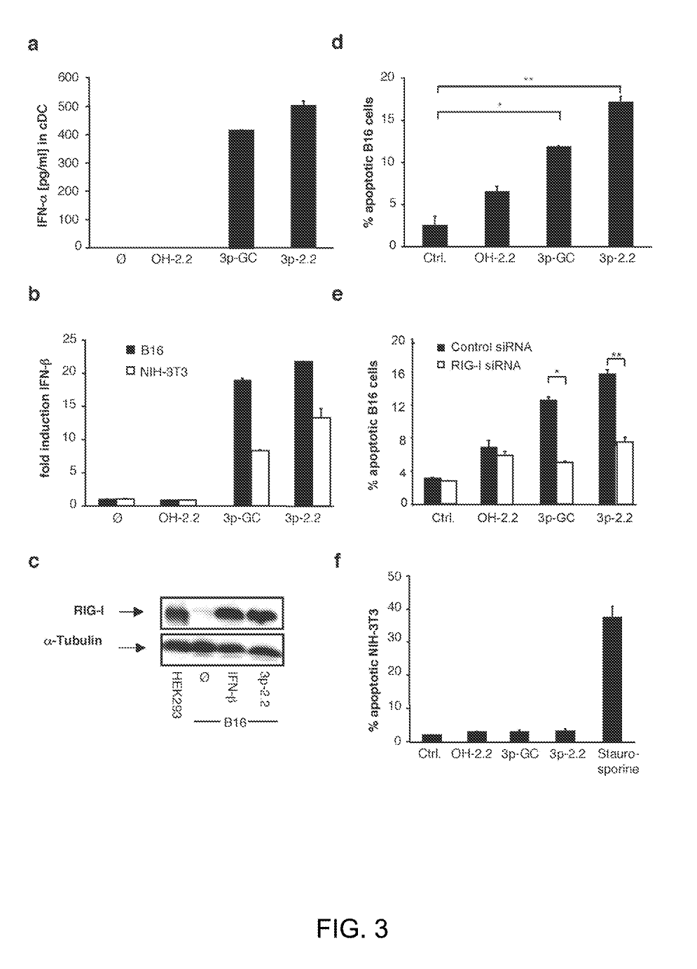

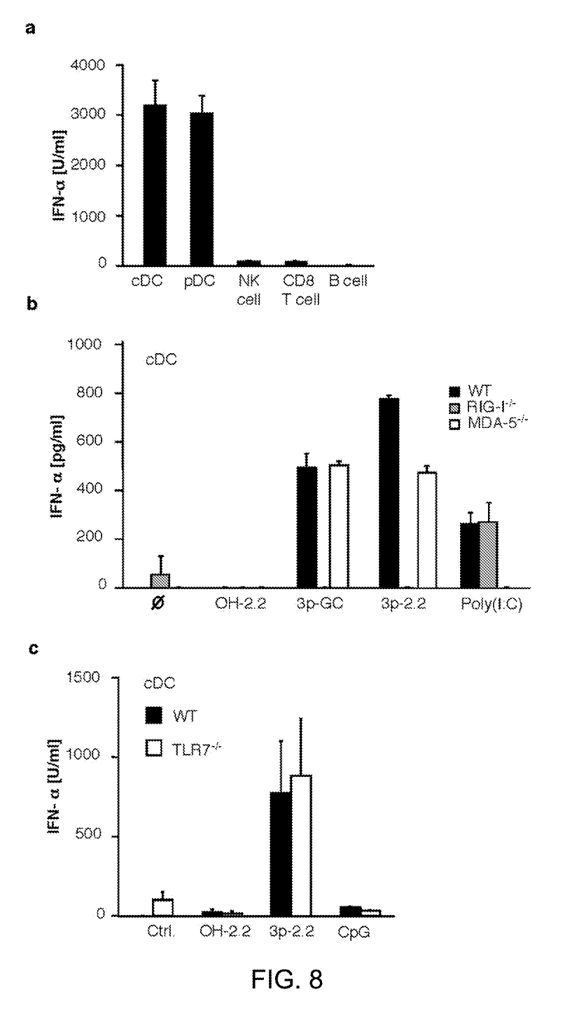

In subsequent experiments we aimed at dissecting the mechanisms leading to innate immune activation and investigated the induction of tumor apoptosis by 3p-2.2 on a cellular level in vitro. First we studied stimulation of immune cell subsets. While in plasmacytoid dendritic cells TLR7 activation is sufficient to induce the production of IFN-α, conventional dendritic cells (cDC) produce IFN-α in response to viral infections¹⁹ but not to TLR7 activation. We examined the ability of cDC and other purified immune cell subsets to produce IFN-α in response to 3p-siRNA. Both 3p-GC (3pRNA but no bcl-2 gene silencing) and 3p-2.2 (3pRNA plus bcl-2 gene silencing) induced similar amounts of IFN-α in cDC, while OH-2.2 (no 3pRNA but bcl-2 gene silencing) was inactive (FIG. 3a). B cells, NK cells and T cells showed no IFN-α response to 3pRNA (FIG. 8a). Studies with dendritic cells isolated from mice genetically deficient for TLR 7 or the cytosolic helicases MDA-5 or RIG-I confirmed that the induction of IFN-α in cDC by 3p-2.2 and 3p-GC depended on the presence of RIG-I but not MDA-5 or TLR7 (FIGS. 8b and c).

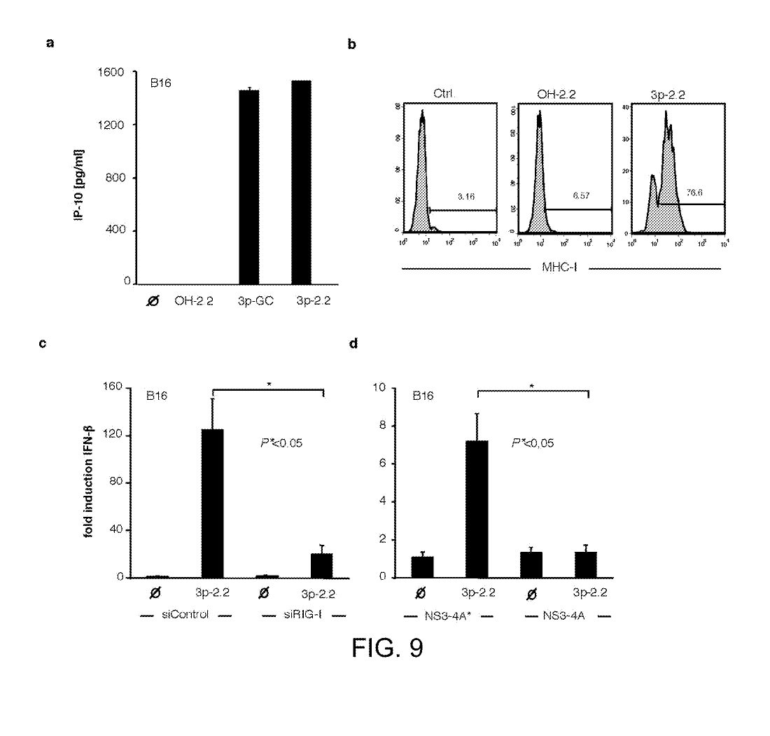

Next, non-immune cells were examined. Since RIG-I is broadly expressed in many cell types^{20,21}, we examined direct induction of type I IFNs in B16 melanoma cells and in NIH-3T3 fibroblasts. 3p-2.2 or 3p-GC stimulated similar levels of IFN-β promoter reporter gene activity both in B16 cells and NIH-3T3 fibroblasts, while both cell types did not respond to OH-2.2 (FIG. 3b). Resting B16 melanoma cells expressed only little RIG-I; however RIG-I expression was strongly upregulated in the presence of exogenous IFN-β or 3p-2.2 (FIG. 3c). Besides activation of the IFN-β promoter, B16 cells treated with 3p-2.2 or 3p-GC secreted the chemokine IP-10 (FIG. 9a) and displayed higher MHC class I expression on their cell surface (FIG. 9b). These data indicated that 3p-siRNA is able to induce type I IFNs not only in immune cells (such as cDC) but also directly in tumor cells. Type I IFN induction in B16 tumor cells was RIG-I dependent, since inhibition of RIG-I expression by RIG-1-specific siRNA or by transfection with a NS3-4A (a multifunctional serine protease of hepatitis C virus which specifically cleaves and thereby inactivates IPS-1^{22,23}, also known as Cardif, MAVS or VISA, a key signaling molecule of RIG-I) both eliminated the type I IFN response (FIGS. 9c and 9d).

In addition to the induction of a type I IFN response in B16 melanoma cells, 3p-2.2 was designed to promote the induction of apoptosis via silencing of the anti-apoptotic protein bcl-2 which is overexpressed in B16 melanoma cells. Indeed, 3p-2.2 strongly induced apoptosis in B16 melanoma cells (FIG. 3d). The observation that apoptosis induction with 3p-2.2 was substantially higher than that observed with OH-2.2 (same sequence than 3p-2.2 but no triphosphate at the 5′ ends) suggested that the ability of 3p-2.2 to activate RIG-I directly contributes to apoptosis induction by this molecule. In fact, downregulation of RIG-I by synthetic siRNA (but not control siRNA) reduced apoptosis induction by 3p-2.2 (but not OH-2.2) in B16 melanoma cells and confirmed that RIG-I activation contributes to the induction of apoptosis by 3p-2.2 (FIG. 3e). This conclusion is supported by the pro-apoptotic effect of 3p-GC, a control RNA with the ability to stimulate RIG-I but without bcl-2 gene silencing activity (FIG. 3d-e). Unlike in B16 tumor cells, neither silencing of bcl-2 (OH-2.2) nor activation of RIG-I (3p-GC) nor the combination of the two mechanisms (3p-2.2) was sufficient to induce apoptosis in NIH-3T3 fibroblasts (FIG. 3f). Collectively, these results suggest that apoptosis can be preferentially induced in melanoma cells by downregulation of bcl-2 and by activation of RIG-I and may provide evidence for the tumor-selective activity of our combinatorial treatment approach.

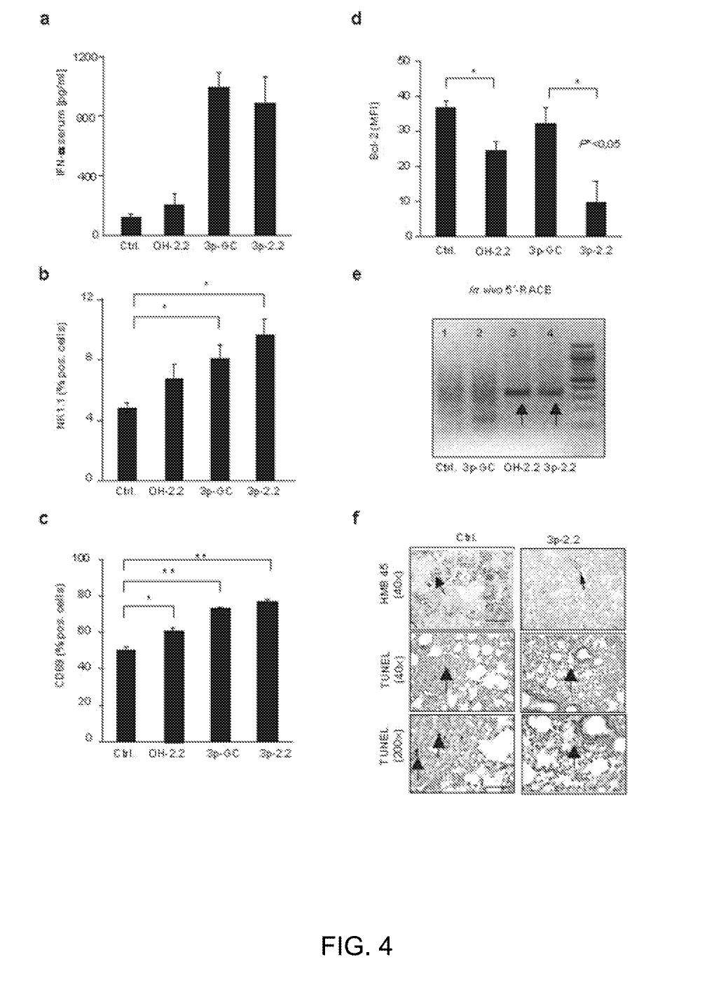

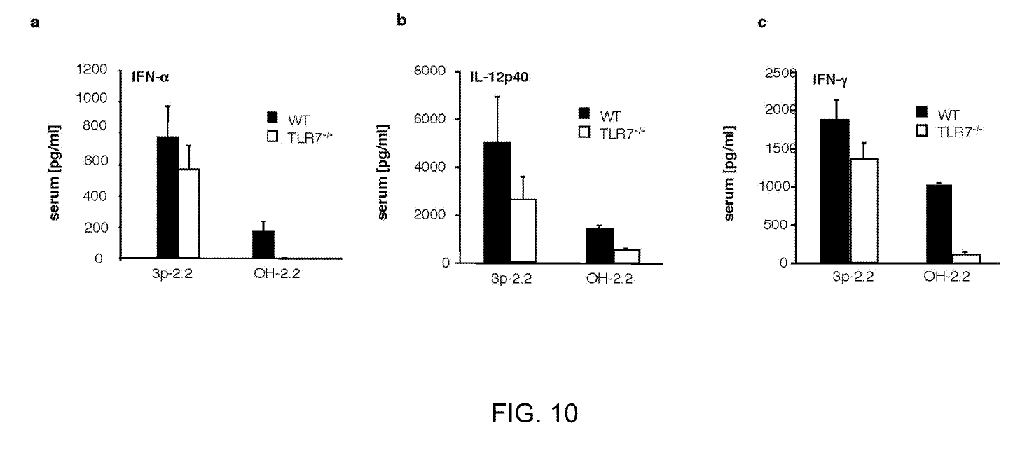

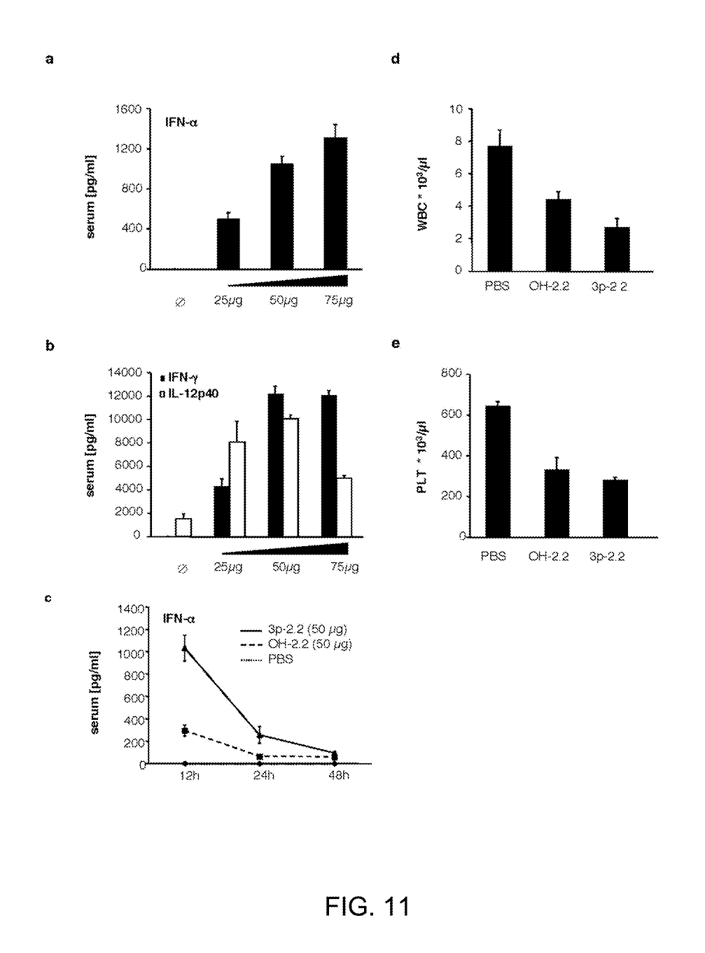

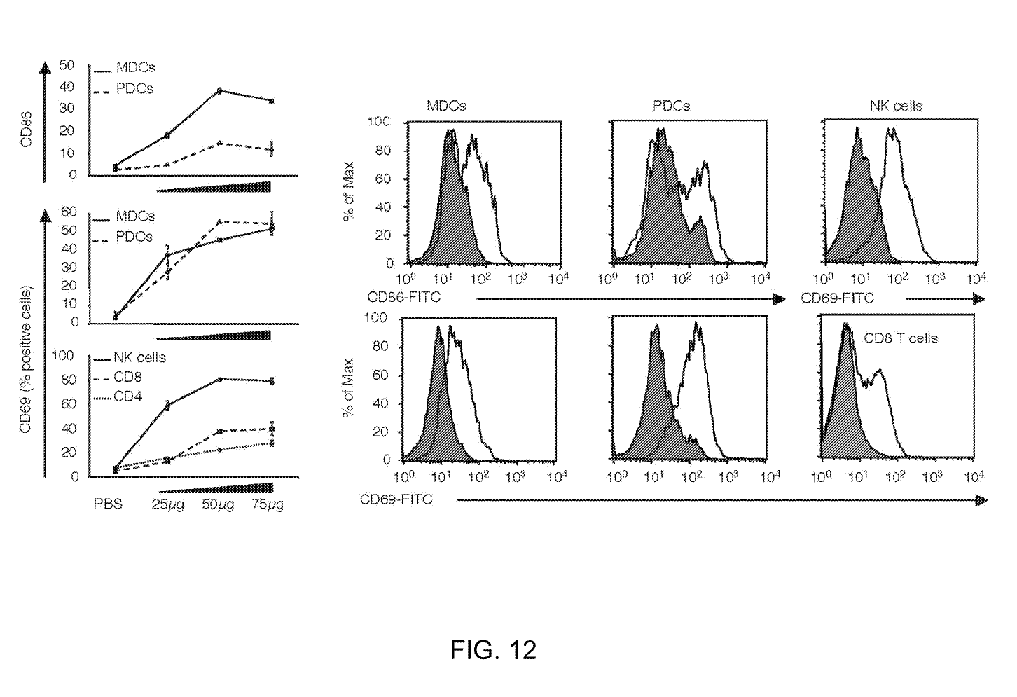

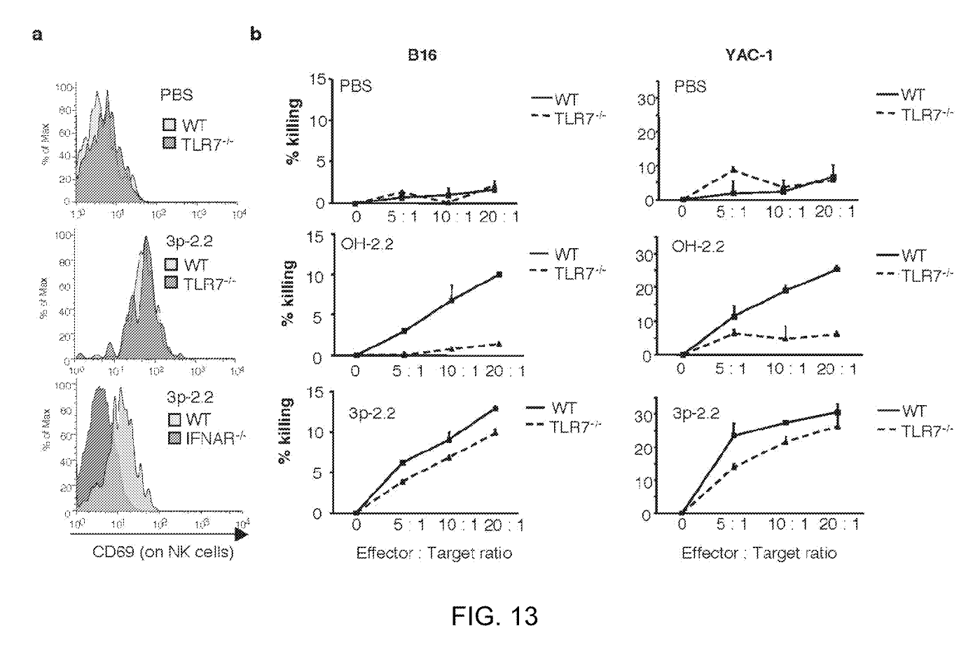

Our results in vivo (FIG. 2) demonstrated that direct induction of apoptosis in tumor cells by bcl-2 silencing and RIG-I activation can not be the only mechanisms that contribute to the therapeutic activity of 3p-2.2. The fact that type I IFNs and NK cells were required for the anti-tumor effect of 3p-2.2 suggested that innate immunity provides another therapeutic component that adds to induction of apoptosis preferentially in tumor cells but not in normal cells. Therefore we further studied the induction of innate immune responses by 3p-2.2 in vivo. Upon intravenous injection, 3p-2.2 induced systemic levels of IFN-α, IL-12p40 and IFN-γ (FIG. 4a, and FIG. 10). The induction of these Th1 cytokines by OH-2.2 (siRNA with no RIG-I ligand activity) was much weaker and completely depended on TLR7. In contrast, both TLR7 and RIG-I contributed to Th1 cytokine induction by 3p-2.2 (FIG. 10). Cytokine production was dose-dependent and showed a rapid decline from 12 h to 48 h (FIG. 11 a-c). Induction of cytokines was associated with leukopenia and thrombocytopenia (FIG. 11 d, e). Analyses of spleen cells demonstrated that treatment with 3p-2.2 also led to potent activation not only of myeloid and plasmacytoid dendritic cells but also of NK cells as well as CD4 and CD8 T cells in a dose-dependent manner in vivo (FIG. 12). Activation of splenic NK cells following treatment with 3p-2.2 was observed in both wildtype as well TLR7-deficient mice and strictly depended on the presence of type I IFNs (FIG. 13a). Importantly, splenic NK cells showed tumoricidal activity against B16 melanoma cells directly ex vivo (FIG. 13b). Furthermore, systemic administration of 3p-2.2 in tumor-bearing mice was associated with enhanced recruitment and activation of NK cells in the lungs (FIG. 4b,c).

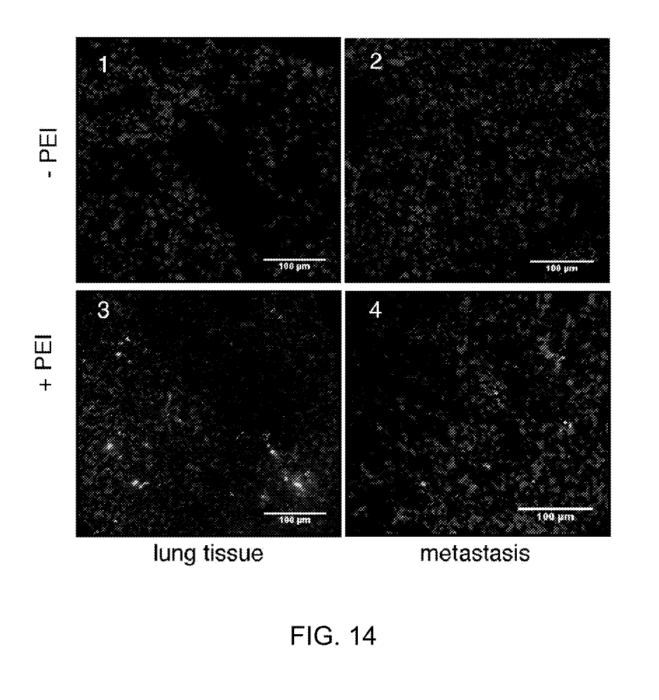

In vivo, confocal microscopy confirmed that fluorescently labeled siRNA reached healthy lung tissue as well as metastases (FIG. 14). In single cell suspensions of lung tissue, tumor cells could be identified by flow cytometry based on their expression of the melanocytic marker gene HMB45. This allows to study the downregulation of bcl-2 selectively in HMB45-positive tumor cells on a single cell level in vivo. We found that bcl-2 was significantly reduced in tumor cells of mice treated with 3p-2.2 and OH-2.2 compared to the corresponding non-target specific control RNA molecules (3p-GC for 3p. 2.2; control-RNA for OH-2.2) (FIG. 4d). Downregulation of bcl-2 by OH-2.2 confirmed that RIG-I ligand activity was not required. Furthermore, the lack of bcl-2 downregulation by 3p-GC confirmed that RIG-I ligand activity was not sufficient; however, RIG-I ligand activity seems to add to the gene silencing activity of siRNA, since 3p-2.2 showed the highest overall activity to downregulate bcl-2. These results in vivo are consistent with an additive effect of gene silencing and RIG-I activation in terms of apoptosis induction in vitro. We also confirmed that the downregulation of bcl-2 is associated with RNAi in vivo, as demonstrated by 5′ rapid amplification of cDNA ends (RACE) analyses (FIG. 4e). Finally, we examined apoptosis on a histological level in lung tissue (FIG. 4f). Tunel staining revealed massive apoptosis in mice treated with 3p-2.2 compared to mice treated with Control-RNA, although the number of HMB45 positive tumor cells (possibly undergoing apoptosis) was much higher in the control-treated animals. Apoptosis was found in lung areas in which remaining tumor cells were detectable based on HMB45 staining (FIG. 4f).

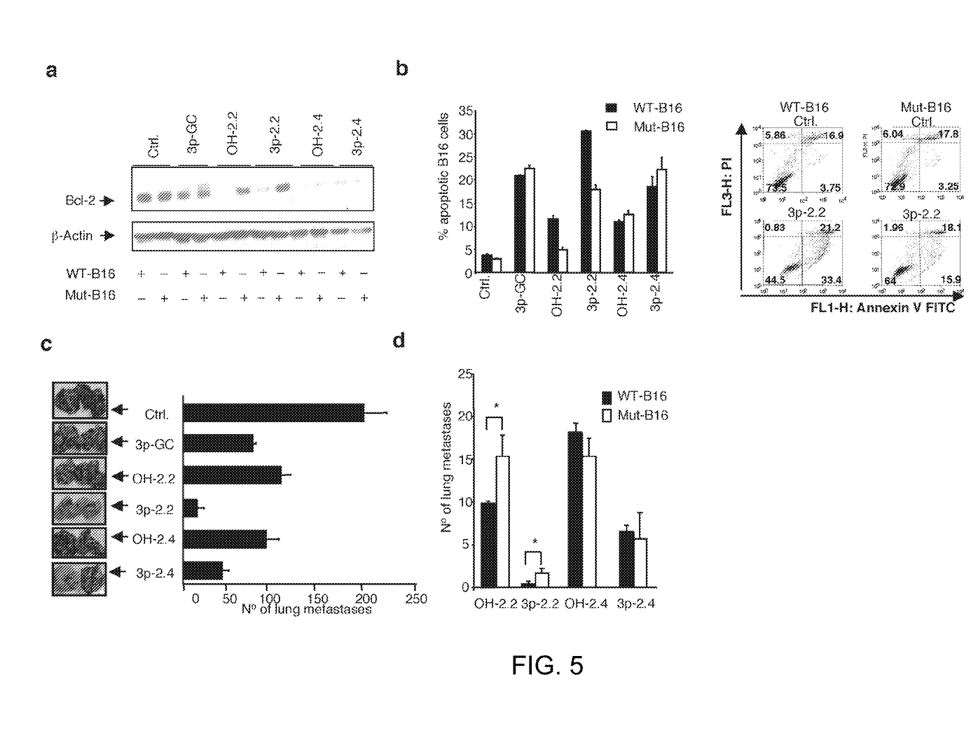

In order to provide more evidence that silencing Bcl-2 plays a significant role in the antitumor effects of 3p-Bcl2-siRNA, we performed siRNA rescue experiments. B16 melanoma cells were stably transduced with a codon-optimized Bcl-2 cDNA carrying a mutation in the target cleavage site of the Bcl-2-specific siRNA 2.2. This prevented siRNA-mediated gene silencing in B16 melanoma cells in vitro following transfection with OH-2.2 as well as 3p-2.2 but not with OH-2.4 or 3p-2.4, another Bcl-2-specific siRNA which targets an alternative sequence (FIG. 5a, Table 1). Mut-B16 were almost completely rescued from the induction of apoptosis following transfection with OH-2.2 and partially rescued for 3p-2.2 (FIG. 5b). Importantly, the second Bcl-2-specific siRNAs OH-2.4 and 3p-2.4 showed anti-tumor efficacy against B16 melanoma lung metastases similar to OH-2.2 and 3p-2.2 (FIG. 5c). In vivo rescue experiments with WT-B16 and Mut-B16 suggested that the therapeutic effect of OH-2.2 and 3p-2.2 was at least in part dependent on Bcl-2 gene silencing in tumor cells (FIG. 5d). Taken together, these results provided important mechanistic insight in the relative contribution of gene silencing and RIG-I activation of 3p-siRNA.

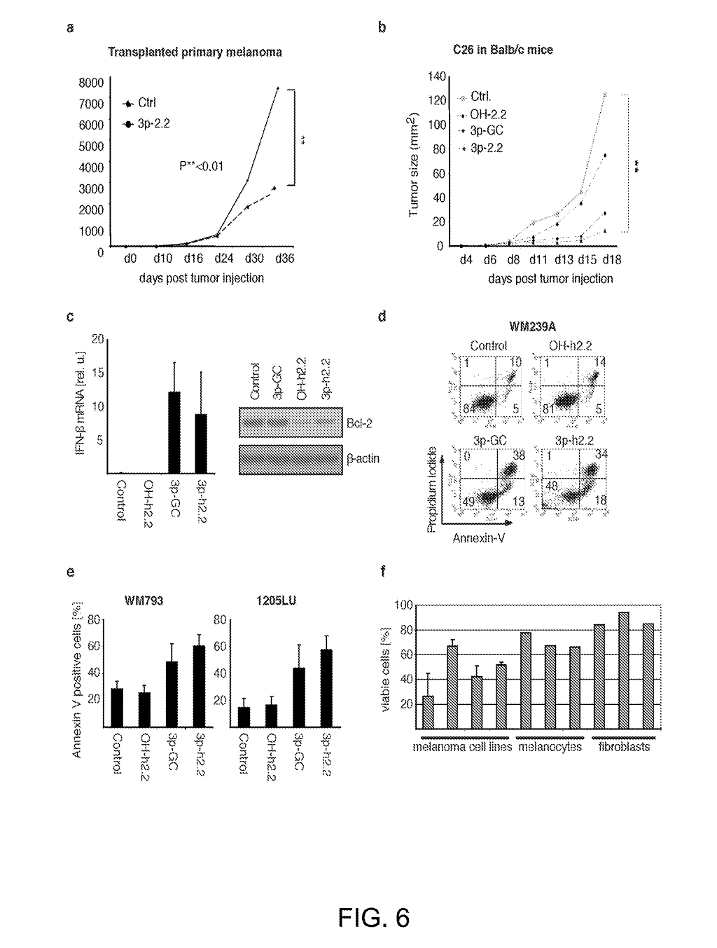

In subsequent experiments we examined the anti-tumor efficacy of 3p-siRNA in other tumor models. We previously established a new genetic melanoma model which is based on important events in the molecular pathogenesis of melanoma and much more closely mimics the clinical situation²⁴. Melanomas derived from the skin of HGF/CDK4^{R24C} mice were serially transplanted to groups of CDK4^{R24C} mice and histopathologically resemble primary cutaneous melanomas. Treatment with intra- and peritumoral injections with 3p-2.2 were performed on days 10, 16, 24 and 30. On day 36 mice were sacrificed. Starting on day 24 a significant delay in tumor growth was observed in 3p-2.2 treated mice (FIG. 6a). In addition, we found that 3p-2.2 (3p-2.2>3p-GC) showed significant anti-tumor efficacy in a colon carcinoma model in Balb/C mice (FIG. 6b).

In order to extend our observations in the human system, we evaluated the effects of 3p-siRNA treatment on human melanoma cells. We designed and tested human anti-bcl-2 siRNA (OH-h2.2 and 3p-h2.2). Treatment of the melanoma cell line 1205 Lu with 3p-h2.2 and 3p-GC, but not with OH-h2.2 or the control RNA was able to induce IFN-β in human melanoma cells (FIG. 6c, left panel). Both OH-h2.2 and 3p-h2.2 strongly reduced bcl-2 protein levels (FIG. 6c, right panel). We then investigated the pro-apoptotic activity of OH-h2.2 and 3p-h2.2 in different human metastatic melanoma cell lines (WM239A; WM793 and 1205Lu). We found that bcl-2 inhibition sensitized WM239A cells to apoptosis (FIG. 6d), but not WM793 or 1205 LU cells (FIG. 6e) suggesting that downregulation of bcl-2 does not play a role in the constitutive resistance to apoptosis in all human melanoma cells. However, similar to B16 cells, transfection of 3p-h2.2 significantly increased the number of apoptotic cells. Strikingly, the pro-apoptotic activity was less pronounced in melanocytes and almost absent in fibroblasts indicating a tumor selective effect (FIG. 6f). In conclusion, our results demonstrate that anti-tumor therapy with 3p-siRNA can be translated to the human system.

The results of this study demonstrate that systemic administration of a siRNA deliberately designed to silence bcl-2 and to activate RIG-I (3p-2.2) strongly inhibits tumor growth reflected by massive apoptosis on a histological level. Our data show that type I IFN and NK cells are required for this response, and that this effect is associated with the induction of systemic Th1 cytokines (IFN-α, IL-12p40, IFN-γ), direct and indirect activation of immune cell subsets, with recruitment and activation of NK cells in lung tissue and with inhibition of bcl-2 in tumor cells in treated mice in vivo.

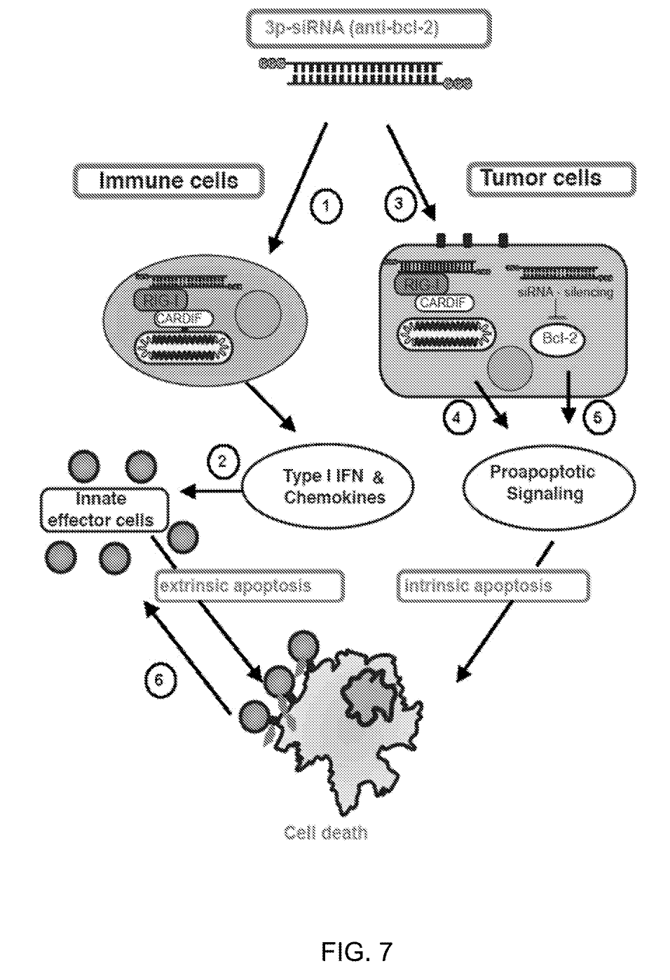

Based on its molecular structure, the combinatorial siRNA molecule used (3p-2.2) contains two clearly distinct functional properties, a) gene silencing and b) RIG-I activation; but a number of biological effects caused by these two properties may cooperate to provoke the beneficial response against the tumor in vivo: a) silencing of bcl-2 may induce apoptosis in cells that depend on bcl-2 overexpression, and via this mechanism may as well sensitize those cells towards innate effector cells²⁵. b) RIG-I is expressed in immune cells as well as in non-immune cells including tumor cells; consequently, activation of RIG-I may lead to direct and indirect activation of immune cell subsets, but also may provoke innate responses directly in tumor cells such as the production of type I IFNs or chemokines. In addition, RIG-I activation may directly induce apoptosis in cells sensitive to RIG-I-mediated apoptosis. All of those biological processes may act in concert to elicit the potent anti-tumor effect seen (for a schematical overview of the potential antitumor-mechanisms elicited by 3p-siRNA see FIG. 7.)

In fact, our data provide experimental evidence that B16 tumor cells express RIG-I and that 3p-2.2 not only silences bcl-2 but also stimulates type I IFN, IP-10, MHC I, and induces apoptosis. Furthermore, in immune cells in vitro, 3p-2.2 acts as a RIG-I ligand exemplified by the stimulation of IFN-α production in myeloid (conventional) dendritic cells. We demonstrate that silencing of bcl-2 in tumor cells does not require RIG-I ligand activity (OH-2.2, same sequence as 3p-2.2 but no triphosphate), and that RIG-I effects are independent of bcl-2 silencing activity (3p-GC, triphosphate but no silencing). Importantly, compared to the respective single activities, the data demonstrate synergistic induction of tumor cell apoptosis in vitro and synergistic inhibition of bcl-2 and induction of apoptosis in the tumor in vivo when both silencing and RIG-I activity are in place (3p-2.2 compared to OH-2.2 or 3p-GC alone).

Although our data confirm that the innate immune system (NK cells, type I IFN) is critically involved in the overall anti-tumor activity in vivo, the relative contribution of innate effector cells on top of direct tumor apoptosis induced by bcl-2 silencing and RIG-I activation is difficult to assess. The lower anti-tumor response in vivo together with the lack of bcl-2 inhibition in tumor cells in vivo by the RIG-I ligand (3p-GC) alone confirm that gene silencing is a key functional property of 3p-2.2. Likewise, the weak overall anti-tumor response to anti-bcl-2 siRNA (OH-2.2) despite strong inhibition of bcl-2 in tumor cells in vivo highlights the importance of the innate contribution. However, each mechanism by itself is not sufficient to effectively suppress tumor growth in vivo. This result is supported by our rescue experiments which showed that apoptosis induced by OH-2.2 depended completely while apoptosis induced by 3p-2.2 depended only in part on bcl-2 gene silencing.

A key question is how systemic administration of the combinatorial RNA molecule 3p-2.2 can result in the tumor specificity observed. Retroorbital injection as performed in this study is considered equivalent to intravenous injection, resulting primarily in systemic distribution of the compound. Fluorescently-labeled RNA complexed with polyethylenimine (PEI) was enriched in lungs but also liver, spleen and kidney (data not shown). Thus, in our study RNA delivery is not targeted to the tumor. Nevertheless, a relative tumor specificity of apoptosis induction is seen in the murine and the human system which may be explained by a cooperation of the following three mechanisms in our approach: first, like in human melanoma, B16 melanoma cells express high levels of bcl-2 nt spontaneous tumor cell apoptosis^{14,16}, while in normal cells all checkpoints of apoptosis are intact and inhibition of bcl-2 alone is not sufficient for apoptosis induction. This is supported by our data comparing B16 tumor cells and NIH-3T3 fibroblasts as well as human melanoma cells and their human counterparts, i.e. human fibroblasts and human melanocytes. Second, in our hands RIG-I activation is sufficient to induce apoptosis in B16 tumor cells and human melanoma cells but not in normal cells such as NIH-3T3 fibroblasts, human fibroblasts and human melanocytes. Third, B16 melanoma cells are much more sensitive to killing by activated NK cells, strongly upregulate MHC I expression and secrete high amounts of IP-10 only after transfection with 3p-siRNA. We therefore hypothesize that RIG-1-mediated activation of the type I IFN system in tumor cells leads to changes on the cell surface that predisposes these cells for NK cell attack and destruction, similar to what was proposed by Stetson and Medzhitov²⁵.

Our studies show that treatment with 3p-siRNA can be extended to other models of tumorigenesis. We were able to demonstrate anti-tumor activity against melanomas derived from primary cutaneous tumors in HGF×CDK4^{R24C} mice. The HGF×CDK4^{R24C} mouse melanoma model resembles the expected clinical situation in melanoma patients much more closely, firstly because melanomas arise as a consequence of genetic alterations similar to those observed in patients and secondly because melanomagenesis can be promoted by UV irradiation. Repeated administration of 3p-2.2 resulted in a significant delay in tumor growth in this model. We also observed a significant anti-tumor efficacy of 3p-siRNA in a syngenic subcutaneous colon carcinoma model in Balb/c mice. Most importantly, we provide evidence that treatment with bcl2-specific 3p-siRNA can be adapted to the human system. A bcl2-specific 3p-siRNA mediated gene silencing as well as RIG-I activation in human melanoma cells promoting the induction of apoptosis, whereas melanocytes and fibroblasts were resistant to apoptosis induction. These results suggest that the principles of the approach presented in this study may have great promise for clinical translation.

The gene silencing activity of the RNA molecule can be directed to any given molecularly defined genetic event that governs tumor cell survival. A combination of siRNA sequences selected for different tumor-related genes is feasible. New targets identified by functional tumor genetics can directly be imported in the approach of combinatorial RNA. This will advance our ability to attack the tumor from different biological angles which we think is required to effectively counteract tumor plasticity and tumor escape. Despite the relative tumor specificity seen in our study, it is assumed that this strategy in the future will be further improved by targeted delivery of the compound to tumor tissue.

Material and Methods

Media and Reagents

RPMI 1640 (Biochrom) supplemented with 10% (v/v) heat-inactivated FCS (Invitrogen Life Technologies), 3 mM L-glutamine, 0.01 M HEPES, 100 U/ml penicillin, and 100 μg/ml streptomycin (all from Sigma-Aldrich) and Dulbecco's modified Eagle's medium (PAN, Aidenbach, Germany) supplemented with 10% fetal calf serum (FCS), 3 mM L-glutamine, 100 U/ml penicillin and 100 μg/ml streptomycin was used. Recombinant murine IFN-β was purchased from Europa Bioproducts LTD. In vivo-jetPEI (#201-50) was purchased from Biomol GmbH (Hamburg, Germany). Staurosporine was purchased from Sigma-Aldrich (S6942).

RNAs

Chemically synthesized RNA oligonucleotides were purchased from Eurogentec (Leiden, Belgium) or MWG-BIOTECH AG (Ebersberg, Germany). For a detailed list of all chemically synthesized RNA oligonucleotides see Table 1. For some experiments PolyA or control-siRNA were used as Control-RNAs (indicated in Table 1). In vitro transcribed RNAs were synthesized according to the manufacturer's instructions using the megashort script kit (Ambion, Huntingdon, UK). For a detailed list of all in vitro transcription templates see Table 2. The templates contained a T7 RNA Polymerase consensus promoter followed by the sequence of interest to be transcribed. For generation of in vitro transcribed double-stranded RNA the DNA templates of the sense and anti-sense strands were transcribed for 6 hours in separate reactions. An extra Guanosin was added at the 5′ end to both the sense and the anti-sense strands in order to transcribe with T7 RNA polymerase. The reactions were then mixed and incubated overnight at 37° C. to anneal the transcribed RNA strands. The DNA template was digested using DNAse-I (Ambion) and subsequently RNAs were purified by phenol:chloroform extraction and alcohol precipitation. Excess salts and NTPs were removed by passing the RNAs through a Mini Quick Spin™ Oligo Column (Roche). Integrity of RNAs was checked via gel electrophoresis.

Cell Culture

Plasmacytoid DC from Flt3-ligand-induced (Flt3-L) bone marrow cultures were sorted with B220 microbeads (Miltenyi Biotec, Bergisch-Gladbach, Germany). Conventional dendritic cells (cDC) were generated by incubating pooled bone marrow cells in the presence of murine GM-CSF (10 ng/ml; R&D Systems, Minneapolis, Minn.). After 7 days, these cultures typically contained more than 80% cDC(CD11c⁺, CD11b⁺, B220⁻). For some experiments B cells were isolated from spleens of wild-type mice by MACS using the mouse B cell isolation kit and CD19 microbeads (Milteny Biotec). Untouched NK cells and CD 8 T cells were sorted from spleens using the NK cell isolation and the CD8 T Cell Isolation Kit (Milteny Biotec). Viability of all cells was above 95%, as determined by trypan blue exclusion and purity was >90% as analyzed by FACS. Murine primary cells were cultivated in RPMI (PAN) supplemented with 10% fetal calf serum (FCS), 4 mM L-glutamine and 10-−5 M mercaptoethanol. Murine B16 cells (H-2b) were cultivated in Dulbecco's modified Eagle's medium (PAN) supplemented with 10% fetal calf serum (FCS), 2 mM L-glutamine, 100 U/ml penicillin and 100 μg/ml streptomycin. NIH-3T3 cells (murine fibroblasts) were cultivated in Dulbecco's modified Eagle's medium (PAN) supplemented with 10% fetal calf serum (FCS), 2 mM L-glutamine, 100 U/ml penicillin and 100 μg/ml streptomycin. C26 is colon cancer cell line (Cell Lines Service, Heidelberg) syngeneic to BALB/c mice and was maintained in DMEM supplemented with 10% FCS, 2 mM L-glutamine, 100 g/ml streptomycin and 1 IU/ml penicillin at 37° C. and 5% CO₂.

Transfection of RNA In Vitro

For siRNA experiments B16 cells were seeded in 24-well flat-bottom plates, respectively. At a confluency of 50-70% cells were incubated for 24 hours with 5′-triphosphate siRNA (1 μg/ml), synthetic siRNA (1 μg/ml), or Control siRNA (1 μg/ml). RNAs were transfected with Lipofectamine 2000 or Lipofectamine RNAimax (both Invitrogen) according to the manufacturer's protocol. DC and immune cell subsets were transfected with 200 ng of nucleic acid with 0.5 μl of Lipofectamine in a volume of 200 μl. After 24 h the supernatants were collected for analysis of cytokine secretion by enzyme-linked immunosorbent assay (ELISA), and cells were harvested for flow cytometric analysis.

Cytokine Measurements

Concentrations of murine IFN-γ and IL-12p40 in the culture supernatants or sera were determined by ELISA according to the manufacture's instructions (BD PharMingen, San Diego, Calif.). Murine IFN-α was analyzed using the mouse IFN-α ELISA kit (PBL Biomedical Laboratories, PBL #42100-2, New Brunswick, N.J.). For some experiments, murine IFN-α was measured according to the following protocol: monoclonal rat anti-mouse IFN-α (clone RMMA-1) was used as the capture Ab, and polyclonal rabbit anti-mouse IFN-α serum for detection (both PBL Biomedical Laboratories) together with HRP-conjugated donkey anti-rabbit IgG as the secondary reagent (Jackson ImmunoResearch Laboratories). Mouse rIFN-α (PBL Biomedical Laboratories) was used as a standard (IFN-α concentration in IU/ml). Mouse IP-10 (R&D Systems) was determined by ELISA according to the manufacturer's instructions.

Transfection and IFN-β Reporter Assay

For monitoring transient IFN-β activation by 5′-triphosphate siRNA murine B16 cells were seeded in 24-well plates. At a confluency of 70%, B16 cells were transfected using high molecular weight (25 kDa) polyethylenimine (PEI; Sigma,) with 200 ng of a reporter plasmid (pIFN-β-luc DAM/DCM), 200 ng of a normalization plasmid (expressing Renilla-Luc) and the indicated expression plasmids giving a total of 1.5 μg DNA/well. A PEI:DNA ratio of 1.5:1 was used. In some experiments Lipofectamine 2000 (Invitrogen) for co-transfection of synthetic siRNAs with the indicated expression plasmids was used according to the manufacturer's protocol.

16 hours after transfection culture medium was aspirated, the cells were washed once with PBS and stimulated with different ligands for the indicated time points. The supernatant was collected and the cells were washed again with PBS containing 10 mM EDTA and lysed in 100 μl of Promega lysis buffer (Promega). 20 μl of each sample were mixed with 20 μl of Luciferase Detection Reagent (Luciferase Assay Kit, Biozym Scientific GmbH, Oldendorf, Germany) and analyzed for luciferase activity with a microplate luminometer (LUMIstar, BMG Labtechnologies). To measure Renilla luciferase activity, 20 μl lysate was incubated with 20 μA of Renilla substrate (Coelenterazine; Promega). Luciferase activity values were normalized to Renilla activity of the same extract.

Plasmids

IFN-β-Luc reporter plasmids, wild-type pPME-myc NS3-4A (NS3-4A), pPME-myc MutNS3-4A (NS3-4A*; containing an inactivating Serin 139 to Ala mutation) were kindly provided by T. Maniatis and J. Chen. RIG-I and the empty control vector were kindly provided by T. Fujita¹⁰. The renilla-luciferase transfection efficiency vector (phRLTK) was purchased from Promega. cDNA encoding WT murine Bcl-2 (mBc1-2/pcDNA) was provided by C. Borner (Institute of Molecular Medicine and Cell Research, Albert-Ludwigs-University of Freiburg, Germany)

Rescue Experiments

To create mismatches in the target site of murine Bcl-2 we introduced two central silent mutations by site-directed mutagenesis according to the manufacturer's instructions (Site-directed mutagenesis kit; Stratagene; La Jolla, USA) The following primers were used:

mBcl-2 2015 forward (5′ to 3′):
(SEQ ID NO: 1)
CTATATGGCCCCAGCATGAGGCCTCTGTTTGATTTCTCC;
mBCL-22015 reverse (5′ to 3′):
(SEQ ID NO: 2)
GGAGAAATCAAACAGAGGCCTCATGCTGGGGCCATATAG.

cDNA encoding WT murine Bcl-2 served as template. The cDNAs of WT-Bcl-2 and Mutated-Bcl-2 were subsequently sequenced for confirmation (data not shown). For production of lentiviral particles WT-Bcl-2 and Mut-Bcl-2 were cloned by PCR from the pcDNA3 vector into the cloning site of the lentiviral expression vector pLVUB-puromycin and transfected in HEK293T cells together with the 3^{rd} generation packaging plasmids (pMDL g/P RRE; pRSV-REV) and the envelope plasmid (pVSV-G) using Lipofectamine-2000. On day 3 supernatant was collected and used for transduction of B16 cells. Infected cells were selected for insertion of the construct with puromycin (1 μg/ml) for three weeks.

In Vitro and In Vivo Race

Total RNA of B16 cells (in vitro) or from pooled metastatic lungs of the indicated groups (in vivo) was purified using Tryzol reagent (Invitrogen), subsequently DNase treated and applied to RNeasy clean-up procedure (QIAGEN). bug of RNA preparation from pooled samples was ligated to GeneRacer adaptor without prior treatment:

(SEQ ID NO: 3)
(5′-CGACUGGAGCACGAGGACACUGACAUGGACUGAAGGAGUAGAAA)

Ligated RNA was reverse transcribed using a gene-specific primer. To detect cleavage product, 2 rounds of consecutive PCR were performed using primers complementary to the RNA adaptor and mBc12 mRNA (GR5′ and Rev 1 or Rev.2 for the 1^{st} PCR round; GRN5′ and RevN—for the nested PCR). Amplified products were resolved by agarose gel electrophoresis and visualized by ethidium bromide staining The identity of specific cleavage products was confirmed by cloning of the PCR product and sequencing of individual clones.

Western Blotting

Adherent and non-adherent cells were lysed in a buffer containing 50 mM Tris; pH 7.4, 0.25M NaCl, 1 mM EDTA, 0.1% Triton X-100, 0.1 mM EGTA, 5 mM Na3VO4, 50 mM NaF and protease inhibitors (Complete, Mini, EDTA-free, Roche) and samples were separated by SDS-PAGE and transferred to a nitrocellulose membrane (Amersham-Biosciences, UK) by semi-dry electroblotting. Polyclonal rat anti-RIG-I (kind gift of Dr. Elisabeth Kremmer, Institute of Molecular Immunology, GSF—National Research Center for Environment and Health, Munich, Germany) or anti-bcl-2 (Santa Cruz, sc-7382) antibodies were incubated at 4° over night and detected via a peroxidase-conjugated anti-rat or anti-rabbit antibody (Amersham-Biosciences). Bands were visualized by chemiluminescence according to the manufacturer's protocol (ECL Kit; Amersham-Biosciences).

Flow Cytometry

At the time points indicated, surface antigen staining was performed as described 17. Fluorescence-labeled monoclonal antibodies (mAbs) against B220, CD11c, NK1.1, CD4, CD8, CD69, CD86, MHC-I (H2-Kb) and appropriate isotype control antibodies were purchased from BD Pharmingen (Heidelberg, Germany). Goat anti-Mouse IgG1 FITC was purchased from Santa Cruz (sc-2078). To determine bcl-2 Expression in vivo, single cell suspensions of metastatic lungs were prepared. These single cell suspensions were fixed and permeabilized using 2% paraformaldehyde and 0.5% Saponin and ultimately incubated with monoclonal melanosome antibody, clone HMB45 (anti-melanosome, HMB45; Dako Cytomation) for 20 min on ice. Subsequently, cells were washed and incubated with goat anti-mouse IgG1 FITC Ab (Santa Cruz; sc-2012) for 20 min on ice. Thereafter, cells were washed again and PE-conjugated bcl-2-Ab (Santa Cruz, sc-7382-PE) was added. After 20 min of incubation cells were analyzed by flow cytometry. Bcl-2 expression of melanoma cells in lungs was quantified by gating on HMB45 positive cells and detecting bcl-2-PE fluorescence. Flow cytometric data were acquired on a Becton Dickinson FACS Calibur. Data were analyzed using CellQuest software (Becton Dickinson, Heidelberg, Germany).

Assessment of NK Cytolytic Activity.

Cytolytic activity of purified NK cells derived from 3p-2.2-treated mice was determined by 51Cr-release assay. Mice were i.v. injected with 50 μg of 3p-2.2. After 16 h, mice were killed and NK cells were purified from spleens with DX5 (anti-CD49b) microbeads (Miltenyi Biotec) according to the manufacturer's recommendations. Target cells (5000/well) were labeled with 51Cr for 4 h at 37° C., then washed and coincubated with effector cells at the indicated effector-to-target cell ratio. Cytotoxicity was determined by measuring the 51Cr radioactivity released in 100 μl of the supernatant harvested from the plate after 16 h of incubation at 37° C. The percentage of specific lysis was calculated by using the formula: % Specific lysis=[(experimental release−spontaneous release)/(total release−spontaneous release)]×100.

Quantification of Apoptotic and Dead Cells

Adherent and supernatant cells were analyzed by staining with FITC-labeled Annexin-V (Roche) and propidium iodide (BD Biosciences) Annexin-V staining was performed according to the manufacturer's instructions. Propidium iodide was added to a final concentration of 0.5 mg/ml and cells were analyzed by flow cytometry and CellQuest software (Becton Dickinson, Heidelberg, Germany). For induction of apoptosis in murine fibroblasts, staurosporine (Sigma-Aldrich) was used at 1 μM.

Quantification of Viable Cells

Viable cells were quantified in six-well dishes utilizing a fluorimetric assay (CellTiter-Blue Cell Viability Assay, Promega, Mannheim, Germany). Viable cells with intact metabolism are determined by their ability to reduce cell-permeable resazurin to fluorescent resorufin. Medium was replaced with 750 ml of culture medium and 150 ml of CellTiter-Blue reagent. After 1 h incubation at 37° C. fluorescence was measured.

Confocal Microscopy

C57BL/6 mice were injected intravenously with FITC labeled RNA (100 μg) complexed with jetPEI (Biomol). After 6 h mice were sacrificed and the lungs were analyzed for uptake of the RNA complexes. Briefly, sections of metastatic lungs or non-diseased lungs were transferred on microscope slides and fixed in acetone for 10 min. Nuclear counterstaining was performed using TOPRO-3 (Molecular Probes). Washing steps were done in Tris-buffered saline and cells were mounted in Vectarshield Mounting Medium (Vector Laboratories). Cells were then analyzed using a Zeiss LSM510 confocal microscope (Carl Zeiss, Germany) equipped with 488 nm-Argon and 633 nm-Helium-Neon lasers.

Mice

RIG-1-, MDA-5-, TLR7-deficient mice were established as described 26, 27. IFNAR-deficient mice were a kind gift of Ulrich Kalinke and were established as described 28, 29. Female C57BL/6 and Balb/c mice were purchased from Harlan-Winkelmann (Borchen, Germany). Mice were 6-12 weeks of age at the onset of experiments. Animal studies were approved by the local regulatory agency (Regierung von Oberbayern, Munich, Germany). HGF/CDK4R24c mice were generated as described 24.

Mouse Studies

For in vivo studies, we injected C57BL/6 mice with 200 μl containing nucleic acids with prior jetPEI-complexation according to the manufacturer's protocol. Briefly, 10 μl of in vivo jetPEI was mixed with 50 μg of nucleic acids at a N:P ration of 10/1 in a volume of 200 μl 5% Glucose solution and incubated for 15 min. Subsequently, the complexes were injected in the retro-orbital or the tail vein. Serum was collected after 6 h unless indicated otherwise. Whole blood was obtained by tail clipping at the indicated time points. Serum was prepared from whole blood by coagulation for 30 min at 37° C. and subsequent centrifugation. Cytokine levels were determined by ELISA.

Engraftment of B16 Melanoma in the Lungs and Depletion of CD8 T Cells and NK Cells In Vivo

For the induction of lung metastases we injected 4×10⁵ B16 melanoma cells into the tail vein. On day 3, 6 and 9 after tumor cell inoculation 50 μg of jetPEI-complexed RNA in a volume of 200 μl was administered by injection into the retro-orbital or the tail vein. 14 days after challenge the number of macroscopically visible melanoma metastases on the surface of the lungs was counted with the help of a dissecting microscope or, in case of massive tumor load, lung weight was determined. Depletion of NK cells and CD8 T cells was performed as described³⁰. Briefly, for neutralization of NK cells TMβ1 mAb was given intraperitoneally 4 days (1 mg) before and 2 (0.2 mg) and 14 (0.1 mg) days after tumor challenge. To neutralize CD8 T cells, the mAb RmCD8-2 was injected intraperitoneally one (0.5 mg) and four days (0.1 mg) before and 4 (0.1 mg) and 14 (0.1 mg) days after tumor inoculation. Experiments were done in groups of four to five mice. For in vivo RACE experiments we injected 4×10⁵ B16 melanoma cells into the tail vein. On day 8 after tumor cell inoculation 150 μg of jetPEI-complexed siRNA was administered by injection in a volume of 200 μl into the retro-orbital vein. 24 h and 48 h after injection of the jetPEI-complexed siRNA mice were sacrificed and lungs were homogenized. Subsequently, total RNA from pooled metastatic lungs of the indicated groups was purified using Tryzol reagent (Invitrogen).

Serial Transplantation of Primary Cutaneous Melanomas Derived from HGF×CDK4R24C/R24C Mice.

Primary melanomas were induced in the skin of HGF×CDK4^{R24C/R24C} mice by neonatal treatment with 7,12-dimethylbenz[a]anthracene (DMBA) as described previously^{24,31}. Progressively growing cutaneous melanomas exceeding 10 mm in diameter were sacrificed, dissociated with sterile scissors and passed through a nylon mesh filter (70 μl) with PBS. Melanoma cells were reinjected in the flank of CDK4^{R24C/R24C} mice and tumor growth assessed weekly by palpation. Transplanted primary HGF×CDK4^{R24C/R24C} melanomas initially developed after about 2 months. Upon serial intracutaneous transplantation, tumors appeared earlier and grew with similar kinetics in different mice. Treatment experiments were performed with groups of 5 mice intracutaneously injected with approximately 10⁵ viable transplanted HGF×CDK4^{R24C/R24C} melanoma cells derived from one transplanted melanoma in the fourth to sixth passage. Tumor growth was monitored weekly by measuring the maximal two bisecting diameters (L=length and W=width) using a vernier sliding jaw caliper. Tumor size was calculated according to the formula Volume=(L×W²)×0.5 and expressed in mm³. Mice with tumors greater than 4000 mm³ were sacrificed.

Induction of C26 Tumors in the Skin

For tumor induction in Balb/c mice, C26 cells were washed in PBS and 2.5×10⁵ cells were injected subcutaneously in the right flank in a volume of 200 μl. Tumor growth was monitored three times a week and expressed as the product of the perpendicular diameters of individual tumors (mm²).

Histopathologic Analyses

Mice were sacrificed and lung tissue samples were fixed in absolute ethanol and embedded in paraffin. Monoclonal antibody against HMB45 (HMB45; Dako Cytomation) was used to identify metastatic tissue. Apoptosis was detected within metastases by the transferase-mediated dUTP nick end-labeling (TUNEL) method according to the manufacturer's instructions (Roche, Mannheim, Germany). Briefly, deparaffinized and rehydrated sections were incubated for 1 h at 37° C. with tailing mix containing 1× tailing buffer, 1 mM CoCl₂, 1 μl of 10×DIG DNA labeling mix and 200 units of terminal transferase (double dist. water added to a total volume of 50 μl). After washing in Tris-buffered saline, sections were incubated for 1 h at room temperature with an alkaline phosphatase-conjugated anti-digoxigenin antibody conjugate (diluted 1:250 in 10% fetal calf serum). The reaction was visualized with nitro blue tetrazolium/5-bromo-4-chloro-3-indolyl phosphate.

Statistical Analyses

Statistical significance of differences was determined by the two-tailed Student's t-test. Differences were considered statistically significant for P<0.05. For the analysis of the tumor experiments we used the non-parametric Mann-Whitney U test to compare the means between two groups. Statistical analysis was performed using SPSS software (SPSS, Chicago, Ill.). P values <0.05 were considered significant.

TABLE 1
Chemically synthesized RNA sequences

| | | | SEQ |
|---|---|---|---|
| | | | ID |
| Name | Type | Sequence 5′ to 3′ | NO. |
| Murine bcl-2 | RNA | AUGCCUUUGUGGAACUAUA | 4 |
| 2.1 sense | | | |
| Murine bcl-2 | RNA | UAUAGUUCCACAAAGGCAU | 5 |
| 2.1 anti-sense | | | |
| Murine bcl-2 | RNA | GCAUGCGACCUCUGUUUGA | 6 |
| 2.2 sense | | | |
| Murine bcl-2 | RNA | UCAAACAGAGGUCGCAUGC | 7 |
| 2.2 anti-sense | | | |
| Murine bcl-2 | RNA | GGAUGACUGAGUACCUGAA | 8 |
| 2.3 sense | | | |
| Murine bcl-2 | RNA | UUCAGGUACUCAGUCAUCC | 9 |
| 2.3 anti-sense | | | |
| PolyA | RNA | AAAAAAAAAAAAAAAAAAA | 10 |
| (used in FIGS. | | | |
| 1c, 2a-d; 4a-d; | | | |
| 4f) | | | |
| Murine RIG-I | RNA | GAAGCGUCUUCUAAUAAUU | 11 |
| Sense | | | |
| Murine RIG-I | RNA | AAUUAUUAGAAGACGCUUC | 12 |
| anti-sense | | | |
| Control siRNA | RNA | UUCUCCGAACGUGUCACGU | 13 |
| Sense (used in | | | |
| FIG. 1a, b, | | | |
| 3d-f, 4e, 5a-d, | | | |
| 6a-e) | | | |
| Control siRNA | RNA | ACGUGACACGUUCGGAGAA | 14 |
| anti-sense | | | |
| (used in FIG. | | | |
| 1a, b, 3d-f, 4e, | | | |
| 5a-d, 6a-e) | | | |
| Murine Bcl-2 | RNA | GGAGAACAGGGTATGATAA | 15 |
| 2.4 sense | | | |
| Murine Bcl-2 | RNA | CCTCTTGTCCCATACTATT | 16 |
| 2.4 Anti-sense | | | |
| Human Bcl-2 | RNA | GCATGCGGCCTCTGTTTGA | 17 |
| h2.2 sense | | | |
| Human Bcl-2 | RNA | CGTACGCCGGAGACAAACT | 18 |
| h2.2 Anti-sense | | | |

TABLE 2
DNA-oligonucleotides (templates) for in
vitro transcription

| | | | SEQ ID |
|---|---|---|---|
| Name | Type | Sequence 5′ to 3′ | NO: |
| Murine bcl-2 | DNA | TCAAACAGAGGTCGCATGCCTATAGTGAGTCG | 19 |
| 2.2 sense | | | |
| Murine bcl-2 | DNA | GCATGCGACCTCTGTTTGACTATAGTGAGTCG | 20 |
| 2.2 anti-sense | | | |
| GC sense | DNA | GGCGCCCCGCCGCGCCCCGCTATAGTGAGTCG | 21 |
| GC anti-sense | DNA | GCGGGGCGCGGCGGGGCGCCTATAGTGAGTCG | 22 |
| Murine BcL-2 | DNA | TTATCATACCCTGTTCTCCCTATAGTGAGTCG | 23 |
| 2.4 sense | | | |
| Murine Bcl-2 | DNA | GGAGAACAGGGTATGATAACTATAGTGAGTCG | 24 |
| 2.4 Anti-sense | | | |
| Human Bcl-2 | DNA | TCAAACAGAGGCCGCATGCCTATAGTGAGTCG | 25 |
| h2.2 sense | | | |
| Human Bcl-2 | DNA | GCATGCGGCCTCTGTTTGACTATAGTGAGTCG | 26 |
| h2.2 Anti-sense | | | |

TABLE 3
Primers used for 5′-RACE

| | | | SEQ ID |
|---|---|---|---|
| Name | Application | Sequence 5′ to 3′ | NO. |
| cDNA | cDNA synthesis | GTT CAT CTG AAG TTT CCA GCC TTT G | 27 |
| GR 5′ | 5′RACE product | CGACTGGAGCACGAGGACACTGA | 28 |
| | forward PCR | | |
| | primer, 1st round | | |
| GRN | 5′RACE product | GGACACTGACATGGACTGAAGGAGTA | 29 |
| 5′ | forward PCR | | |
| | primer, nested | | |
| | round | | |
| Rev.1 | 5′RACE product | TCC CTT TGG CAG TAA ATA GCT GAT TCG ACC AT | 30 |
| | reverse PCR | | |
| | primer, 1st round, | | |
| | in vivo samples | | |
| | assay | | |
| Rev.2 | 5′RACE product | AAG TCC CTT CTC CAG TCC ATG GAA GAC CAG | 31 |
| | reverse PCR | | |
| | primer, 1st round, | | |
| | in vitro samples | | |
| | assay | | |
| RevN | 5′RACE product | CTT TGG CAG TAA ATA GCT GAT TCG ACC ATT TGC | 32 |
| | reverse PCR | | |
| | primer, nested | | |
| | round | | |

As used in the specification and the appended claims, the singular forms “a,” “an” and “the” include plural referents unless the context clearly dictates otherwise.

The examples described herein are specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES

1. Hanahan, D. & Weinberg, R. A. The hallmarks of cancer. Cell 100, 57-70 (2000).
2. Bui, J. D. & Schreiber, R. D. Cancer immunosurveillance, immunoediting and inflammation: independent or interdependent processes? Curr Opin Immunol 19, 203-8 (2007).
3. Rubin, B. P., Heinrich, M. G. & Corless, C. L. Gastrointestinal stromal tumour. Lancet 369, 1731-41 (2007).
4. Curiel, T. J. Tregs and rethinking cancer immunotherapy. J Clin Invest 117, 1167-74 (2007).
5. Uno, T. et al. Eradication of established tumors in mice by a combination antibody-based therapy. Nat Med 12, 693-8 (2006).
6. Obeid, M. et al. Calreticulin exposure dictates the immunogenicity of cancer cell death. Nat Med 13, 54-61 (2007).
7. Schlee, M., Hornung, V. & Hartmann, G. siRNA and is RNA: two edges of one sword. Mol Ther 14, 463-70 (2006).
8. Pei, Y. & Tuschl, T. On the art of identifying effective and specific siRNAs. Nat Methods 3, 670-6 (2006).
9. de Fougerolles, A., Vornlocher, H. P., Maraganore, J. & Lieberman, J. Interfering with disease: a progress report on siRNA-based therapeutics. Nat Rev Drug Discov 6, 443-53 (2007).
10. Yoneyama, M. et al. The RNA helicase RIG-I has an essential function in double-stranded RNA-induced innate antiviral responses. Nat Immunol 5, 730-7 (2004).
11. Pichlmair, A. et al. RIG-1-mediated antiviral responses to single-stranded RNA bearing 5′-phosphates. Science 314, 997-1001 (2006).
12. Hornung, V. et al. 5′-Triphosphate RNA is the ligand for RIG-I. Science 314, 994-7 (2006).
13. Yoneyama, M. & Fujita, T. Function of RIG-1-like receptors in antiviral innate immunity. J Biol Chem 282, 15315-8 (2007).
14. Miller, A. J. & Mihm, M. C., Jr. Melanoma. N Engl J Med 355, 51-65 (2006).
15. Danial, N. N. & Korsmeyer, S. J. Cell death: critical control points. Cell 116, 205-19 (2004).
16. McGill, G. G. et al. Bcl2 regulation by the melanocyte master regulator Mitf modulates lineage survival and melanoma cell viability. Cell 109, 707-18 (2002).
17. Hornung, V. et al. Sequence-specific potent induction of IFN-alpha by short interfering RNA in plasmacytoid dendritic cells through TLR7. Nat Med 11, 263-70 (2005).
18. Judge, A. D. et al. Sequence-dependent stimulation of the mammalian innate immune response by synthetic siRNA. Nat Biotechnol 23, 457-62 (2005).
19. Kawai, T. & Akira, S. Innate immune recognition of viral infection. Nat Immunol 7, 131-7 (2006).
20. Kato, H. et al. Cell type-specific involvement of RIG-I in antiviral response. Immunity 23, 19-28 (2005).
21. Melchjorsen, J. et al. Activation of innate defense against a paramyxovirus is mediated by RIG-I and TLR7 and TLR8 in a cell-type-specific manner. J Virol 79, 12944-51 (2005).
22. Kawai, T. et al. IPS-1, an adaptor triggering RIG-1- and Mda5-mediated type I interferon induction. Nat Immunol 6, 981-8 (2005).
23. Meylan, E. et al. Cardif is an adaptor protein in the RIG-I antiviral pathway and is targeted by hepatitis C virus. Nature 437, 1167-72 (2005).
24. Tormo, D. et al. Therapeutic efficacy of antigen-specific vaccination and toll-like receptor stimulation against established transplanted and autochthonous melanoma in mice. Cancer Res 66, 5427-35 (2006).
25. Stetson, D. B. & Medzhitov, R. Antiviral defense: interferons and beyond. J Exp Med 203, 1837-41 (2006).
26. Diebold, S. S., Kaisho, T., Hemmi, H., Akira, S. & Reis e Sousa, C. Innate antiviral responses by means of TLR7-mediated recognition of single-stranded RNA. Science 303, 1529-31 (2004).
27. Kato, H. et al. Differential roles of MDA5 and RIG-I helicases in the recognition of RNA viruses. Nature 441, 101-5 (2006).
28. Muller, U. et al. Functional role of type I and type II interferons in antiviral defense. Science 264, 1918-21 (1994).
29. Kamphuis, E., Junt, T., Waibler, Z., Forster, R. & Kalinke, U. Type 1 interferons directly regulate lymphocyte recirculation and cause transient blood lymphopenia. Blood 108, 3253-61 (2006).
30. Mocikat, R. et al. Natural killer cells activated by MHC class 1(low) targets prime dendritic cells to induce protective CD8 T cell responses. Immunity 19, 561-9 (2003).
31. Tormo, D. et al. Rapid growth of invasive metastatic melanoma in carcinogen-treated hepatocyte growth factor/scatter factor-transgenic mice carrying an oncogenic CDK4 mutation. Am J Pathol 169, 665-72 (2006).
32. Besch, R., Berking, C., Kammerbauer, C. & Degitz, K. Inhibition of urokinase-type plasminogen activator receptor induces apoptosis in melanoma cells by activation of p53. Cell Death Differ 14, 818-29 (2007).

1. A method of determining whether a double stranded RNA (dsRNA) silences gene expression in a cell in vivo by an RNA interference (RNAi) mechanism, wherein the dsRNA comprises at least two sequences that are complementary to each other, and wherein a sense strand comprises a first sequence, and an antisense strand comprises a second sequence, which comprises a region of complementarity to an mRNA expressed in a mammal, wherein the region of complementarity is 19 to 20 nucleotides in length, and wherein the dsRNA further comprises a 5-triphosphate, the method comprising:
(i) providing an RNA sample isolated from the mammal, wherein the mammal was previously administered the dsRNA; and
(ii) performing 5′-rapid amplification of cDNA ends (5′RACE) to detect the cleavage site of the mRNA in the RNA sample; wherein if the mRNA detectable by 5′RACE is cleaved at the predicted site, then the dsRNA is determined to silence gene expression by an RNAi mechanism.

2. (canceled)

3. (canceled)

4. (canceled)

5. (canceled)

6. (canceled)

7. A method of determining whether a double stranded RNA (dsRNA) silences gene expression in cells in vitro by an RNA interference (RNAi) mechanism, wherein the dsRNA comprises at least two sequences that are complementary to each other, and wherein a sense strand comprises a first sequence, and an antisense strand comprises a second sequence, which comprises a region of complementarity to an mRNA expressed in the cells, wherein the region of complementarity is 19 to 20 nucleotides in length, and wherein the dsRNA further comprises a 5′-triphosphate, the method comprising:
(i) providing an RNA sample isolated from the cells, wherein the cells were previously contacted with the dsRNA; and
(ii) performing 5′-rapid amplification of cDNA ends (5′RACE) to detect the cleavage site of the mRNA in the RNA sample; wherein if the mRNA detectable by 5′RACE is cleaved at the predicted site, then the dsRNA is determined to silence gene expression by an RNAi mechanism.

8. (canceled)

9. (canceled)

10. (canceled)

11. (canceled)

12. A method of eliciting anti-tumor activity in a tumor, comprising administering a short interfering RNA (siRNA) to a mammal,
wherein the siRNA comprises triphosphate groups at the 5′ ends,
wherein the siRNA silences an anti-apoptotic gene, and
wherein the siRNA activates helicase RIG-I.

13. The method of claim 12, wherein the tumor is a metastatic tumor.

14. The method of claim 12, wherein the tumor is a melanoma.

15. The method of claim 12, wherein the siRNA induces production of type I IFN or chemokines.

16. The method of claim 12, wherein the siRNA induces production of IFN-alpha, IFN-gamma, IL-12p40, Th1 cytokines, IP-10, or MHC I.

17. The method of claim 12, wherein the siRNA induces apoptosis.

18. The method of claim 17, wherein the apoptosis is Cardif-independent apoptosis.

19. The method of claim 12, wherein the anti-apoptotic gene is overexpressed in tumor cells.

20. The method of claim 12, wherein the anti-apoptotic gene is Bcl-2 gene.

21. The method of claim 12, wherein activation of RIG-I activates an immune cell.

22. The method of claim 21, wherein the immune cell is an NK cell, a CD8 T cell, or a CD4 T cell.

23. The method of claim 12, wherein RIG-I activation sensitizes tumor cells to extrinsic apoptosis.

24. The method of claim 12, wherein RIG-I activation sensitizes tumor cells to intrinsic apoptosis.

25. The method of claim 12, wherein the anti-tumor activity is inhibition of tumor growth.

26. The method of claim 12, wherein the mammal is a mouse.

27. The method of claim 12, wherein said administering is intravenous.
